(19) United States
(12) Patent Application Publication
(10) Pub. No.: US 2016/0377618 A1
(43) Pub. Date: Dec. 29, 2016

(54) PCSK9 QUANTIFICATION BY IMMUNODETECTION

(71) Applicant: True Health Diagnostics LLC, Frisco, TX (US)

(72) Inventor: Philip Guadagno, Vashon Island, WA (US)

(21) Appl. No.: 15/193,316

(22) Filed: Jun. 27, 2016

Related U.S. Application Data
(60) Provisional application No. 62/185,204, filed on Jun. 26, 2015.

(51) Int. Cl. G01N 33/573; G01N 27/447; G01N 33/92

(52) U.S. Cl. CPC G01N 33/573; G01N 33/92; G01N 27/44778; G01N 2800/32; G01N 2333/96441; G01N 2800/044; G01N 2800/52; G01N 2333/775

(57) ABSTRACT

The present disclosure relates to, among other things, systems for detecting the level of specific lipoprotein-bound level of PCSK9, and optionally free PCSK9, lipoprotein or portions thereof and/or PCSK9 unbound lipoproteins present in a biological sample. The present invention also relates to methods of assessing the level of specific lipoprotein-bound level of PCSK9, and optionally free PCSK9, Apo B and/or PCSK9 unbound lipoproteins present in a biological sample, determining whether a subject is at increased risk for cardiovascular disease and monitoring the risk for developing cardiovascular disease.

### PRIORITY CLAIM

This application claims priority to U.S. Provisional Patent Application No. 62/185,204, filed Jun. 26, 2015, the entire contents of which are incorporated herein by reference and relied upon.

### SEQUENCE LISTING

The instant application contains a Sequence Listing which has been submitted electronically in ASCII format and is hereby incorporated by reference in its entirety. Said ASCII copy, created on Jun. 20, 2016, is named 114784-8052.US01_SL.txt and is 18,116 bytes in size.

### TECHNICAL FIELD

The present invention relates to the field of immunological assays, and particularly detection of lipoprotein-bound PCSK9, free PCSK9, Apo B, and/or unbound lipoprotein by immunological detection.

### BACKGROUND

Cardiovascular disease (CVD) is the primary factor in mortality in the western world and a major problem that continues to require innovative testing and treatments. CVD is a general term for diseases of the heart and circulatory system that can be chronic and/or acute conditions such as atherosclerosis, coronary heart disease, cerebrovascular disease, peripheral arterial disease, stroke, heart failure, and heart attack.

Despite advances in the study of CVD, it remains insufficiently understood. A wide variety of risk factors for CVD, including collective contribution of cardiometabolic disorders, such as type I and type II diabetes, insulin resistance, and beta cell disorders, and also dyslipidemia, hyperlipidemia, inflammation, protein and lipid factors, contribute to chronic and acute CVD.

Advanced diagnostics provide opportunities to address the problem through the detailed assessment of factors like lipoproteins, their subdivisions including HDL and LDL, their proteome and lipidome, like apolipoprotein B and apolipoprotein(a), and their interaction and binding partners that may factor into lipoprotein and cholesterol metabolism, like LDL receptors and proprotein convertase subtilisin kexin type 9 (PCSK9). Analysis of these factors can provide a more complete understanding a particular patient's cardiovascular condition, therapeutic needs, and effectiveness.

PCSK9 in particular is important to measure in the context of cholesterol and lipid assessment. It is currently unresolved whether serum PCSK9 concentration is predictive of CVD in patients and/or modifies CVD risk factors. PCSK9 modifies and has an inverse correlation with LDL-C concentration. Therapeutics directed to the modification of pathways containing PCSK9 to treat cardiovascular conditions and their underlying cholesterol disorders that involve PCSK9 have been developed or are under development. In particular, monoclonal antibodies that block PCSK9 have been approved for clinical use for severe hypercholesterolemia.

PCSK9 is a cholesterol-modifying chaperone protein, manufactured in the liver and released into the serum that binds to low-density lipoprotein receptors (LDLR). LDLR on the surface of hepatocytes bind and internalize LDL particles and chaperone proteins, cholesterol, triglycerides, and phospholipids from serum. When PCSK9 binds a receptor on a hepatocyte LDLR, the particle and receptor are destroyed. This results in reduced LDLR activity and increased levels of plasma LDL cholesterol. Without binding and subsequent destruction, the receptor would be recycled and continue the action of removing LDL cholesterol from blood.

The correlation between PCSK9 and lipoproteins, in particular Lp(a) is not well understood. In the cells and blood, apolipoprotein B (Apo B) on lipid particles and PCSK9 bind together. However, not all Apo B containing particles have a PCSK9 bound to the particles and not all PCSK9 will be bound; they remain free. PCSK9 is known to also increase Apo B levels through poorly understood pathways which may involve free- or bound-PCSK9. Elevated Apo B concentration in patients is, along with elevated LDL-C, associated with some cardiovascular disease (CVD). Thus, the levels of PCSK9 and Apo B are important to accurate determination of cardiovascular health.

Methods for measurement of PCSK9 are currently insufficient to assess the lipoprotein-bound and free PCSK9 and unbound lipoprotein portions in a sample accurately. Such measurements can better indicate the harmful and active portions of PCSK9 in a subject. In particular, no assays in the art provide detailed information about PCSK9 in a living subject's blood that is vital to assessment and treatment of CVD in patients. Similarly, there is a lack of assays for the assessment of PCSK9 inhibitor drug efficacy. This disclosure is directed to overcoming these and other deficiencies in the art.

### SUMMARY

This disclosure is predicated on discovery of assays and methods for providing detailed information about PCSK9 levels and PCSK9 complexes. In one aspect, the disclosure relates to gel electrophoresis systems for detecting a level of PCSK9 bound and/or unbound to lipoprotein particles present in a biological sample comprising: a gel substrate to receive a biological sample; and an anti-PCSK9 antibody, at least one anti-lipoprotein antibody, or both.

Another aspect of the disclosure relates to immunoassay systems for detecting a level of PCSK9 bound and/or unbound to lipoprotein particles present in a biological sample comprising: an anti-lipoprotein antibody immobilized on a solid substrate; and an anti-PCSK9 antibody, wherein the anti-PCSK9 antibody is bound to a distinct signal producing molecule capable of producing or causing production of a distinguishable detectable signal.

Another aspect of the disclosure relates to methods for measuring improvement or worsening of a patient undergoing treatment for hypercholesterolemia, said method comprising: obtaining a biological sample from the patient; measuring the amount of lipoprotein-bound PCSK9, free PCSK9, or both in the biological sample; measuring the amount of lipoprotein-bound Apo B, free Apo B, or both in the biological sample; optionally measuring an amount of triglyceride-rich lipoprotein (TRL) in the biological sample; generating a ratio of bound:free PCSK9, of bound PCSK9:Apo B, and/or of bound PCSK9:TRL; and comparing the ratio(s) generated to a reference level(s).

Yet another aspect of the disclosure relates to methods for assessing risk of a patient developing cardiovascular disease or progression of cardiovascular disease, said method comprising: obtaining a biological sample from the patient; measuring an amount of lipoprotein-bound PCSK9, free PCSK9, or both in the biological sample; measuring an amount of lipoprotein-bound Apo B, free Apo B, or both in the biological sample; optionally measuring an amount of triglyceride-rich lipoprotein (TRL) in the biological sample; generating a ratio of bound:free PCSK9, of bound PCSK9:Apo B, and/or of bound PCSK9:TRL; and comparing the ratio(s) to a reference level(s).

In some embodiments, the systems further comprise a non-specific protein dye.

In some embodiments, the anti-PCSK9 antibody, at least one anti-lipoprotein antibody, or both are bound to distinct signal producing molecules capable of producing or causing production of a detectable signal and wherein each detectable signal is distinguishable from the other detectable signal(s).

In some embodiments, the systems further comprise a device for detecting the anti-PCSK9 antibody, the at least one anti-lipoprotein antibody, a detectable signal attached thereto, a non-specific protein dye, or any combination thereof. In some embodiments, the device quantitates the level of specific lipoprotein-bound PCSK9, free PCSK9, PCSK9-free lipoprotein, PCSK9-bound lipoprotein, or any combination thereof based on said detecting.

In some embodiments, the anti-lipoprotein antibody is selected from the group consisting of anti-lipoprotein (a), anti-apolipoprotein B (Apo B), and anti-apolipoprotein-a (apo-a).

In some embodiments, the systems further comprise a reagent capable of interacting with a signal producing molecule, wherein the signal producing molecule produces the detectable signal upon contact with the reagent.

In some embodiments, the detectable signal is detectable by fluorometric means.

In some embodiments, the systems are non-denaturing.

In some embodiments, the methods further comprise evaluating the level of improvement or worsening in hypercholesterolemia of the patient by a decreasing or increasing ratio.

In some embodiments, worsening of the patient undergoing treatment for hypercholesterolemia is determined when there is a greater level of PCSK9 than the reference level, the ratio of bound:free PCSK9 is greater than the reference level, the ratio of bound PCSK9:Apo B is greater than the reference level, or the ratio of bound PCSK9:TRLs is greater than the reference level.

In some embodiments, the methods further comprise evaluating the level of risk of developing or progression of cardiovascular disease by a decreasing or increasing ratio.

In some embodiments, the risk of developing cardiovascular disease or progression of cardiovascular disease is determined to be higher or worsening when there is a greater level of PCSK9 than the reference level, the ratio of bound:free PCSK9 is greater than the reference level, the ratio of bound PCSK9:Apo B is greater than the reference level, or the ratio of bound PCSK9:TRLs is greater than the reference level.

In some embodiments, the lipoprotein-bound PCSK9, free PCSK9, or both is measured via an immunoassay comprising at least an anti-PCSK9 detection antibody and a signal-producing molecule physically bound to the anti-PCSK9 detection antibody and quantifying PCSK9 via signal detected from the signal-producing molecule.

In some embodiments, the lipoprotein-bound Apo B, free Apo B, or both is measured via an immunoassay comprising at least an anti-Apo B detection antibody and a signal-producing molecule physically bound to the anti-Apo B detection antibody and quantifying Apo B via signal detected from the signal-producing molecule.

In some embodiments, the anti-PCSK9 antibody binds to an epitope of PCSK9 that comprises at least one residue or a plurality of residues consisting of SEQ ID NO 1, SEQ ID NO 2, SEQ ID NO 3.

One aspect of the disclosure relates to a gel electrophoresis system for detecting the level of specific PCSK9 bound to lipoprotein particles present in a biological sample. The system includes a gel substrate to receive a biological sample and an anti-PCSK9 antibody. Each antibody that binds a PCSK9 protein or a portion thereof. The antibody is detectable by a non-specific protein dye. The system also includes a device for detecting the dye, wherein said detecting indicates the presence or absence of said specific PCSK9 proteins bound to lipoprotein particles in the biological sample.

Another aspect of the disclosure relates to a gel electrophoresis system for detecting the level of specific PCSK9 and apolipoprotein B present in a biological sample. The system includes a gel substrate to receive a biological sample and anti-PCSK9 and anti-Apo B antibodies. The sample is applied to different lanes on the gel and anti-PCSK9 antibody is applied to a different lane than the anti-Apo B antibodies. The antibodies are detectable by a non-specific protein dye. The system also includes a device for detecting the dye, wherein said detecting indicates the presence or absence of said specific PCSK9 proteins and Apo B in the biological sample.

Another aspect of the disclosure relates to a gel electrophoresis system for detecting the level of specific PCSK9 bound to lipoprotein particles present in a biological sample. The system includes a gel substrate to receive a biological sample and an anti-PCSK9 antibody. Each antibody that binds a PCSK9 protein or a portion thereof, where the antibody is bound to a signal producing molecule capable of producing or causing production of a detectable signal. This method also includes contacting the biological sample with the antibody under conditions suitable to form a lipoprotein-antibody-signal producing molecule complex and separating the lipoprotein particles present in the biological sample by depositing the biological sample on an electrophoretic gel and carrying out gel electrophoresis. This method further includes detecting the detectable signal produced by the signal producing molecule of the lipoprotein-antibody-signal producing molecule complex on the electrophoretic gel and determining the level of the specific lipoprotein-bound level of PCSK9, and optionally free PCSK9, Apo B and/or PCSK9 unbound lipoproteins present in the biological sample based on the detecting.

Another aspect of the disclosure relates to a gel electrophoresis system for detecting the level of specific PCSK9 and Apo B bound to lipoprotein particles present in a biological sample. The system includes a gel substrate to receive a biological sample and an anti-PCSK9 antibody and anti-Apo B antibody. Each antibody binds either a PCSK9 protein or Apo B or a portion thereof, where the antibody is bound to a signal producing molecule capable of producing or causing production of a detectable signal. This method also includes contacting the biological sample with the antibody under conditions suitable to form a lipoprotein-antibody-signal producing molecule complex and separating the lipoprotein particles present in the biological sample by depositing the biological sample on an electrophoretic gel and carrying out gel electrophoresis. This method further includes detecting the detectable signal produced by the signal producing molecule of the lipoprotein-antibody-signal producing molecule complex on the electrophoretic gel and determining the level of the specific lipoprotein-bound level of PCSK9, and optionally free PCSK9, Apo B and/or PCSK9 unbound lipoproteins and Apo B present in the biological sample based on the detecting.

Another aspect of the disclosure relates an immunoassay for capture and assessment of bound PCSK9 concentrations on a solid substrate. The system includes a solid substrate with bound anti-Apo B capture antibodies. It further includes anti-PCSK9 detection antibodies, where the detection antibody is bound to a signal producing molecule capable of producing or causing production of a detectable signal. Alternatively, it involves an anti-PCSK9 primary detection antibody and an anti-anti-PCSK9 secondary detection antibody bound to a signal producing molecule. It finally incorporates a biological sample containing lipoproteins, wherein the sample incubated with capture antibodies, and those Apo B-containing lipoproteins are immobilized, then the detection antibodies are incubated with the immobilized lipoprotein particles, binding to PCSK9 moieties. This method further includes detecting the detectable signal produced by the signal producing molecule of the lipoprotein-antibody-signal producing molecule complex or lipoprotein-antibody-antibody-signal producing molecule and determining the level of the specific lipoprotein-bound level of PCSK9, and optionally free PCSK9, Apo B and/or PCSK9 unbound lipoproteins present in the biological sample based on the detecting.

Another aspect of the disclosure relates an immunoassay for assessment of bound PCSK9 concentrations and Apo B concentrations on a solid substrate. The system includes a solid substrate with bound anti-Apo B capture antibodies. It further includes anti-PCSK9 and anti-Apo B detection antibodies, where the antibodies are bound to distinct signal-producing molecules capable of producing or causing production of a detectable signal. It finally incorporates a biological sample containing lipoproteins, wherein the sample is incubated with the capture antibodies, and those Apo B-containing lipoproteins are immobilized, then the detection antibodies are incubated with the immobilized lipoprotein particles, binding to PCSK9 and Apo B moieties. This method further includes detecting the detectable signal produced by the signal producing molecule of the lipoprotein-antibody-signal producing molecule complex and determining the level of the specific lipoprotein-bound level of PCSK9, and optionally free PCSK9, Apo B and/or PCSK9 unbound lipoproteins present in the biological sample based on the detecting.

Another aspect of the disclosure relates an immunoassay for assessment of bound PCSK9 concentrations and Apo B concentrations on a solid substrate. The system includes a solid substrate with bound anti-Apo B capture antibodies. It further includes anti-PCSK9 and anti-Apo B primary detection antibodies, and anti-anti-PCSK9 and anti-anti-Apo B secondary detection antibodies bound to a signal producing molecule. It finally incorporates a biological sample containing lipoproteins, wherein the sample is incubated with the capture antibodies, and those Apo B-containing lipoproteins are immobilized, then the detection antibodies are incubated with the immobilized lipoprotein particles, binding to PCSK9 and Apo B moieties. Anti-anti-PCSK9 and anti-anti-Apo B antibodies are incubated with the antibody-lipoprotein complexes. This method further includes detecting the detectable signal produced by the signal producing molecule of the lipoprotein-antibody-antibody-signal producing molecule complex and determining the level of the specific lipoprotein-bound level of PCSK9, and optionally free PCSK9, Apo B and/or PCSK9 unbound lipoproteins present in the biological sample based on the detecting.

Another aspect of the disclosure relates an immunoassay for assessment of bound PCSK9 and free PCSK9 concentrations and alternately Apo B concentrations on a solid substrate. The system includes a solid substrate with bound anti-PCSK9 capture antibodies. It further includes anti-PCSK9 and/or anti-Apo B primary detection antibodies bound to a signal producing molecule, and alternately anti-anti-PCSK9 and/or anti-anti-Apo B secondary detection antibodies bound to a signal producing molecule if the primary antibody is not. It finally incorporates a biological sample containing lipoproteins, wherein the sample is incubated with the capture antibodies, and those PCSK9-containing lipoproteins are immobilized, then the detection antibodies are incubated with the immobilized lipoprotein particles, binding to PCSK9 and Apo B moieties. Anti-anti-PCSK9 and anti-anti-Apo B antibodies may additionally be incubated with the antibody-lipoprotein complexes. This method further includes detecting the detectable signal produced by the signal producing molecule of the lipoprotein-antibody- or lipoprotein-antibody-antibody-signal producing molecule complex and determining the level of the specific lipoprotein-bound PCSK9 and free proteins and total triglyceride-rich lipoproteins present in the biological sample based on the detecting.

The foregoing assays may be done independently or in conjunction to determine levels of free- and bound-PCSK9 and total triglyceride rich (Apo B-containing) lipoproteins for calculations of ratios. Additionally, an immunoassay for determination of total PCSK9 levels, as known in the art, may be done in conjunction with any of the previous assays to facilitate ratio calculations.

The purpose of the disclosure is to measure the lipoprotein-bound level of PCSK9, and optionally free PCSK9, Apo B and/or PCSK9 unbound lipoproteins, in order to assess the contributions of each to cardiovascular risk profiles. This assessment of biological activity of patient PCSK9 and Apo B measurement has clinical utility because a healthcare provider can interpret the test results in order to decide and act upon the best clinical treatment for a given patient to lower their LDL and thus cardiovascular risk by various therapeutic means. Preferably, the system and method described herein results in a measurement of bound:unbound PCSK9 concentrations in a biological sample. The ratio and/or absolute bound PCSK9 levels may be compared to a population-based threshold or to a previous measurement of a patient in a series of samples to determine cardiovascular disease risk, progression to cardiovascular disease, or response to a therapeutic drug.

### BRIEF DESCRIPTION OF THE DRAWINGS

FIG. 1 shows the presence of apolipoprotein B (Apo B) (left) and PCSK9 (right) in the same sample by immunological detection on a gel. PCSK9 is observable inside the circles inset into the figure. PCSK9 is found at the same position on the gel as Apo B, indicating its presence on the same lipoparticles as Apo B.

FIGS. 2A-2I show quantitative scans of gels indicating the presence of various proteins and particles including PCSK9 for distinct samples.

### DETAILED DESCRIPTION

The present disclosure relates to, among other things, immunological methods for capture and detection of lipoproteins bound to PCSK9 proteins. The lipoprotein-bound level of PCSK9, and optionally free PCSK9, Apo B and/or PCSK9 unbound lipoproteins present are preferably measured in intact lipid particles in a biological sample. The system includes at least one PCSK9-binding antibody, which may alternately detect or capture intact lipoprotein-PCSK9 complexes. The system further comprises a method for detection of a captured lipoprotein-PCSK9 complex, via optically observable dye saturation, fluorescent labeling or another optically-detectable method known in the art. The present invention also relates to methods of assessing the level of specific lipoprotein-bound level of PCSK9, and optionally free PCSK9, Apo B and/or PCSK9 unbound lipoproteins present in a biological sample, determining whether a subject is at increased risk for cardiovascular disease, and monitoring the risk for developing cardiovascular disease.

Additionally, the present invention relates to a gel electrophoresis system for detecting the level of specific lipoprotein-bound level of PCSK9, and optionally free PCSK9, Apo B and/or PCSK9 unbound lipoproteins present in a biological sample. The system includes a gel substrate to receive a biological sample, and at least one PCSK9-binding antibody. In some cases, the antibody may be bound to a signal producing molecule capable of producing or causing production of a detectable signal. The system also includes a device for detecting the detectable signal. The system described may be described as Lipo-immunofixation electrophoresis (Lipo-IFE) and is discussed further in US Publication No. 20140243431A1 (Guadagno et al.), and U.S. Pat. No. 9,005,419 (Guadagno et al.), incorporated herein by reference in their entireties.

Additionally the invention relates to an immunoassay system for detecting the level of specific lipoprotein-bound level of PCSK9, and optionally free PCSK9, Apo B and/or PCSK9 unbound lipoproteins present in a biological sample. The lipoprotein-bound level of PCSK9, and optionally free PCSK9, Apo B and/or PCSK9 unbound lipoproteins present are preferably measured in intact lipid particles in a biological sample. The system includes a capture antibody immobilized on a solid substrate, and at least one PCSK9 binding antibody. In some cases, the antibody may be bound to a signal producing molecule capable of producing or causing production of a detectable signal. The system also includes a device for detecting the detectable signal.

Suitable biological samples or biosamples according to the invention include human biological matrices, urine, plasma, serum, and human lipoprotein fractions. For example, the sample may be fresh blood or stored blood or blood fractions. The sample may be a blood sample expressly obtained for the assays of this invention or a blood sample obtained for another purpose which can be subsampled for use in accordance with the methods according to the invention. For instance, the biological sample may be whole blood. Whole blood may be obtained from the subject using standard clinical procedures. The biological sample may also be plasma. Plasma may be obtained from whole blood samples by centrifugation of anti-coagulated blood. The biological sample may also be serum. The sample may be pretreated as necessary by dilution in an appropriate buffer solution, concentrated if desired, or fractionated by any number of methods including but not limited to ultracentrifugation, fractionation by fast performance liquid chromatography (FPLC), or precipitation. Any of a number of standard aqueous buffer solutions, employing one of a variety of buffers, such as phosphate, Tris, or the like, at physiological to alkaline pH can be used.

The term “Lipid(s)” means a group of naturally occurring molecules that are soluble in fat and insoluble in water and includes fatty acids, waxes, sterols, fat-soluble vitamins (such as vitamins A, D, E, and K), monoglycerides, diglycerides, triglycerides, phospholipids, lysophospholipids and others. Cholesterol and triglycerides are important fats (lipids) in the blood.

The term “Cholesterol” means a compound of the sterol type found in most, if not all, body tissues, including erythrocytes. Cholesterol and its derivatives are important constituents of cell membranes and precursors of other steroid compounds, bile acids and vitamins, but high concentrations in the blood are thought to promote atherosclerosis. Cholesterol, triglycerides, phospholipids and proteins are the principle components of lipoproteins, and their concentrations and compositions vary among the types of lipoproteins, serving various metabolic functions, some of which are distinctly favorable for avoiding cardiovascular disease, such as HDL, which facilitates removal of cholesterol from the bloodstream and unfavorable, such as LDL, which move lipids into artery walls.

The term “Dyslipidemia” means a condition of elevated LDL cholesterol levels or low levels of HDL cholesterol. It is known to be an important factor coronary heart disease and stroke.

The term “Hyperlipidemia” means elevated lipid levels in the blood.

The term “Hypercholesterolemia” means an excess of cholesterol in the bloodstream relative to some desired level. In some cases, this may pertain to cholesterol levels in serum. In some cases, the term may account for other risk factors that are known to one with skill in the art.

Dyslipidemia, Hyperlipidemia, and Hypercholesterolemia are all forms of “cholesterol related disorders” and include, without restriction, any one or more of the following: hypercholesterolemia, heart disease, metabolic syndrome, diabetes, coronary heart disease, stroke, cardiovascular diseases, Alzheimer's disease and general dyslipidemias, which can be manifested, for example, by an elevated total serum cholesterol, elevated LDL, elevated triglycerides, elevated VLDL, and/or low HDL. Some non-limiting examples of primary and secondary dyslipidemias that can be detected through measurement of bound and free PCSK9 either alone, or in combination with one or more other analytes include metabolic syndrome, diabetes mellitus, familial combined hyperlipidemia, familial hypertriglyceridemia, familial hypercholesterolemia, including heterozygous hypercholesterolemia, homozygous hypercholesterolemia, familial defective apolipoprotein B-100; polygenic hypercholesterolemia; remnant removal disease, hepatic lipase deficiency; dyslipidemia secondary to any of the following: dietary indiscretion, hypothyroidism, drugs including estrogen and progestin therapy, beta-blockers, and thiazide diuretics; nephrotic syndrome, chronic renal failure, Cushing's syndrome, primary biliary cirrhosis, glycogen storage diseases, hepatoma, cholestasis, acromegaly, insulinoma, isolated growth hormone deficiency, and alcohol-induced hypertriglyceridemia. Detection and measurement of bound and free PCSK9 can also be useful in management, monitoring, and measuring response to treatment for atherosclerotic diseases, such as, for example, coronary heart disease, coronary artery disease, peripheral arterial disease, stroke (ischaemic and hemorrhagic), angina pectoris, or cerebrovascular disease and acute coronary syndrome, myocardial infarction. In some embodiments, the measurement of bound and free PCSK9 is useful in influencing methods of reducing the risk of: nonfatal heart attacks, fatal and non-fatal strokes, certain types of heart surgery, hospitalization for heart failure, chest pain in patients with heart disease, and/or cardiovascular events because of established heart disease such as prior heart attack, prior heart surgery, and/or chest pain with evidence of clogged arteries.

Apolipoprotein B (Apo B-100 and Apo B-48) is the protein component of LDL. One molecule of Apo B is present in the phospholipid layer of each LDL. Over 90% of the LDL particle is composed of Apo B. Apo B functions to solubilize cholesterol within the LDL complex, which in turn increases the transport capacity of LDL for subsequent deposit of LDL cholesterol on the arterial wall. The deposit contributes to cardiovascular disease. Apo B is also a protein component of chylomicrons, VLDL, IDL, and Lp(a). Apo B is a large amphipathic helical glycoprotein with 2 isoforms: Apo B-100 (synthesized in the hepatocytes) and Apo B-48 (the structural protein of chylomicrons). Chylomicrons contain Apo B-48 while other lipoprotein particles that contain Apo B contain Apo B-100.

Apo B modulates the activity of enzymes that act on lipoprotein particles, maintains the structural integrity of the lipoprotein particle complex, and facilitates the uptake of lipoprotein particles by acting as ligands for specific cell-surface receptors. Enzymes that act on lipoprotein particles include but are not limited to lipoprotein lipase, lecithin-cholesterol acyltransferase, hepatic-triglyceride lipase, and cholesterol ester transfer protein. Elevated levels of Apo B are found in hyperlipoproteinemia. Apo B-100 is absent in forms of abetalipoproteinemia. High levels of Apo B-100 may be present in hyperlipoproteinemia, acute angina, and myocardial infarction. Apo B-48 stays in the intestine in chylomicron retention disease.

It is well established that increased plasma concentration of Apo B-containing lipoprotein particles is associated with an increased risk of developing atherosclerotic disease. Case control studies have found plasma Apo B concentrations to be more discriminating than other plasma lipids and lipoprotein particles in identifying patients with coronary heart disease (CHD). See De Backer et al., “European Guidelines on Cardiovascular Disease Prevention in Clinical Practice. Third Joint Task Force of European and other Societies on Cardiovascular Disease Prevention in Clinical Practice,” Eur Heart J 24:1601-1610 (2003); Walldius & Jungner, “Apolipoprotein B and Apolipoprotein A-I: Risk Indicators of Coronary Heart Disease and Targets for Lipid-modifying Therapy,” J intern Med 255(2): 188-205 (2004); Walldius, et al., “The Apo B/apoA-I ratio: A Strong, New Risk Factor for Cardiovascular Disease and a Target for Lipid-Lowering Therapy—A Review of the Evidence,” J Intern Med. 259(5):493-519 (2006); Yusuf et al., “Effect of Potentially Modifiable Risk Factors Associated with Myocardial Infarction in 52 Countries (the INTERHEART Study): Case-control Study,” Lancet 364: 937-52 (2004), which are hereby incorporated by reference in their entirety). The utility of Apo B in determining CHD risk has been confirmed by prospective studies, although the extent to which Apo B concentrations were better than serum lipids in predicting risk was variable. Apo B is a component of all atherogenic or potentially atherogenic particles, including very low density lipoprotein particles (VLDL-P), intermediate density lipoprotein particles (IDL-P), low density lipoprotein particles (LDL-P), and lipoprotein(a) particles (Lp(a)-P), and each particle contains one molecule of Apo B. Therefore, Apo B provides a direct measure of the number of atherogenic lipoprotein particles in the circulation. Total Apo B is not homogeneous. Total Apo B will be influenced by its presence of Apo B in the various particles above. Measuring total Apo B alone without separating the particles does not indicate with which particle it is associated.

There is now a clear consensus that Apo B is more strongly predictive of cardiovascular disease (CVD) than low density lipoprotein cholesterol (LDL-C) and a recent consensus conference report from the American Diabetes Association (ADA) and the American College of Cardiology (ACC) recognizes the importance of measurement of Apo B (see Kannel et al., “Cholesterol in the Prediction of Atherosclerotic Disease,” Ann Intern Med 90:85-91 (1979) and Jeyarajah et al., “Lipoprotein Particle Analysis by Nuclear Magnetic Resonance Spectroscopy,” Clin Lab Med 26: 847-70 (2006), which are hereby incorporated by reference in their entirety). An elevated level of Apo B and LDL-P signifies that an individual has increased risk for cardiovascular disease. An elevated level of Apo B and Lp(a)-P signifies that an individual has increased risk for cardiovascular disease.

PCSK9 is bound at least to Apo B-containing lipoprotein LDL, which is the major metabolic pathway for PCSK9 function, as described further herein. Due to the importance of Apo B and related particles (triglyceride-rich lipoproteins, or TRLs) in the trafficking of cholesterol and lipids, the PCSK9-Apo B numerical relationship has significance for the efficiency of cholesterol elimination in a patient.

Lipoprotein particle profiles are different for different individuals and for the same individual at different times. Chylomicrons are produced in the intestine and transport digested fat to the tissues. Lipoprotein lipase hydrolyzes triacylglycerol to form fatty acids. Chylomicrons are one of the largest buoyant particles. VLDL is formed from free fatty acids upon metabolism of chylomicrons in the liver. Lipoprotein lipase hydrolyzes triacylglycerol to form fatty acids. IDL is the unhydrolyzed triacylglycerol of VLDL. IDL becomes LDL due to hepatic lipase. HDL plays a role in the transfer of cholesterol to the liver from peripheral tissues. HDL is synthesized in the liver and intestines.

LDL particles bind to LDL receptors. Upon receptor binding, LDL is removed from the blood. Cells use cholesterol within the LDL for membranes and hormone synthesis. LDL deposits LDL cholesterol on the arterial wall which contributes to cardiovascular disease. LDL causes inflammation when it builds up inside an artery wall. Macrophages are attracted to the inflammation and turn into foam cells when they take up LDL, causing further inflammation. Smaller, denser LDL contains more cholesterol ester than the larger, buoyant LDL.

Low-density lipoprotein cholesterol, (LDL-C), has been used for measurement for assessing cardiovascular risk. However, due to the variability of HDL-C, Apo B is a better measure of circulating LDL particle number (LDL-P) and therefore a more reliable indicator of risk than that traditional LDL-C because there is 1:1 stoichiometry of Apo B and LDL particles. The sum of total Apo B includes but is not limited to the Apo B complement of LDL-P (large buoyant particles and small dense particles), plus VLDL plus IDL plus Lp(a) plus chylomicrons. Measurement of Apo B levels and associated lipoprotein particles provides additional information on the risk of atherosclerotic heart disease beyond that of the individual measurements or the traditional LDL-C assays. Measurement of fasting plasma insulin levels and LDL particle size also provide useful information.

PCSK9 is manufactured in the liver and released into the serum. Through mass action, binding takes place between Apo B on lipid particles and PCSK9. When the lipid particle is taken up by the receptor, the PCSK9 causes destruction of the receptor, which would be recycled if the PCSK9 were not present on the lipid particle. Not all Apo B containing particles will have a PCSK9 bound to the particles and not all PCSK9 will be bound and remain free. Thus, the independent levels and ratios of PCSK9 and Apo B are important for accurate determination of cardiovascular health.

The study and development of PCSK9 as a therapeutic target for cholesterol related disorders and therefore, CVD prevention, has exploded since its discovery in 2003. For example, on the NCBI PubMed literature database, the terms “PCSK9 inhibitor” produces 243 results in the last 5 years, and only 40 results for the 5 years prior. While the entire metabolic pathway of PCSK9 function and metabolism remains incomplete, the success in blocking PCSK9 activity via antibody therapeutics has shown tremendous benefits from hypercholesterolemia patients. Three major drug manufacturers, Sanofi®, Regeneron®, and Amgen® have monoclonal antibody drugs, alirocumab, described in Dadu et al., “Lipid Lowering with PCSK9 Inhibitors,” Nat Rev Cardiology advance online publication 24 June, incorporated herein in its entirety, and evolocumab, described in U.S. Pat. No. 8,030,457, U.S. Pat. No. 8,563,698, U.S. Pat. No. 8,829,165, and U.S. Pat. No. 8,859,741, all incorporated herein in their entirety in the final stages of regulatory approval as of 2015, all with exceptional performance in reducing the 60-80% of remaining CVD risk that is unaddressed by statins alone. Pfizer® additionally has a monoclonal antibody drug, bococizumab, also described in Dadu et al. and a small-molecule inhibitor in early stage regulatory trials. A small-molecule inhibitor may be useful for intermediate conditions of hypercholesterolemia and more advanced conditions, such as Familial Hypercholesterolemia may be candidates for an injectable antibody therapeutic.

Additional PCSK9-targeting therapeutics may be chosen from:

| Name | Company | Therapeutic Type |
|---|---|---|
| Alirocumab | Regeneron/Sanofi | Monoclonal antibody |
| Evolocumab | Amgen | Monoclonal antibody |
| LGT209 | Novartis | Monoclonal antibody |
| RG7652 | Roche/Genentech | Monoclonal antibody |
| Bococizumab | Pfizer | Monoclonal antibody |
| BMS-962476 | Bristol-Myers Squibb | Adnectin |
| ALN-PCS | Alnylam | RNA interference |

The assay of the current invention may be used to monitor treatment effectiveness of any one or more of these therapeutics independently, or in conjunction with other therapies, such as statins, anti-inflammatories, lifestyle modification or other treatment options described further herein.

In one instance, the evaluation of PCSK9 concentration on separate lipoprotein particles may facilitate the recommendation of statin, small-molecule PCSK9 inhibitor, or anti-PCSK9 antibody/vaccine methods of treatment. In some cases, the anti-PCSK9 antibody/vaccine methods of treatment will involve alirocumab, evolocumab or bococizumab. In another instance, the evaluation of PCSK9 levels and distribution facilitates the recommendation of a particular one therapeutic of alirocumab, evolocumab or bococizumab. In another instance, the evaluation of PCSK9 levels and distribution facilitates the recommendation of dosage levels of statin, PCSK9 small-molecule inhibitor, or anti-PCSK9 antibody/vaccine. In another instance, the evaluation of PCSK9 levels and distribution facilitates the recommendation of a change in therapeutic medication.

PCSK9 has a complex metabolic pathway and effect on lipoprotein metabolism that is not fully explained as driving LDLR degradation, although that is believed to be its major function. For example, PCSK9 inhibition also affects lipoprotein concentrations that are not dependent on LDLR removal. The PCSK9-Lp(a) relationship is similarly not understood. PCSK9 interacts with other lipoprotein-related targets such as VLDLR and CD36. There is also evidence that PCSK9 affects the levels of triglyceride-rich lipoproteins. Another common element to triglyceride-rich lipoproteins, LDL, VLDL, IDL, and Lp(a) is apolipoprotein B. The ability to measure both PCSK9 and Apo B by particle type and as attached to a particle is a critical element to further understanding of the mechanisms for PCSK9 regulation of the lipoproteins, and their application to human health. Shapiro et al., “Targeting PCSK9 for Therapeutic Gains,” Curr Artheroscler Rep 17(19): 1-9 (2015).

Despite the fact that Lp(a) is not cleared by LDLR, its concentration is lowered by anti-PCSK9 antibody therapeutics similarly to LDL. Measurement of PCSK9 levels on the Lp(a) particles is critical to understanding this phenomenon. In one version, the measurement of PCSK9 on Lp(a) involves an electrophoretic gel, wherein the lipoproteins in a sample are separated, an anti-apo(a) antibody targeting a non-Kringle IV₂ portion of the apo(a) and used for quantification of Lp(a) particles, and an anti-PCSK9 antibody, to quantify the levels of PCSK9 on Lp(a) particles. In another embodiment, an ELISA system would include an anti-Apo B capture antibody, and anti-apo(a) detection antibody, and an anti-PCSK9 detection antibody, wherein all Apo B-containing particles are captured, Lp(a) particles are quantified through the use of the anti-apo(a) detection antibody, and PCSK9 proteins are quantified through measurement of the anti-PCSK9 antibodies. In one final embodiment, anti-apo(a) capture antibodies capture Lp(a) particles, anti-Apo B or additional anti-apo(a) antibodies are used to facilitate quantification of the Lp(a) particles, and anti-PCSK9 antibodies are used to quantify the Lp(a)-bound portion of PCSK9.

Measurement of Apo B and PCSK9 content on particle types requires an assay system with multiple dimension that have not been contemplated in the prior art. Immunological methods can be tuned to target the dimensions of interest in ways that other methods, such as ultracentrifugation, NMR, and mass spectrometry cannot. Antibodies can be used to target PCSK9 and/or Apo B generally as polyclonal or monoclonal, or specific fragments of those proteins for more specific orientation if necessary. Electrophoretic methods facilitate a size-, isoelectric character- or charge-based separation of particles prior to or in combination with the antibody-based recognition and modulation. For example, an electrophoretic protocol may separate VLDL, IDL, LDL, and Lp(a) on a gel, an anti-lipoprotein antibody (e.g., anti-Apo B antibody) may then be used to fix the particles in the gel and to facilitate quantitation of the particles, then anti-PCSK9 antibodies may be applied to characterize the particles with attached PCSK9. In that case, the two antibodies would be bound to optically-active, but distinct labels for characterization. Using a comparison with a control sample, the levels of Apo B, therefore particle of each type, may be quantified, and the PCSK9 level may also be quantified, giving an indication of the risk factors in a patient. It may be determined that a low level of PCSK9 to LDL makes the patient a poor candidate for anti-PCSK9 therapy.

PCSK9 levels are known to increase in a feedback to statin therapy in patients, thereby moderating the effect of statin treatment. Statin treatment increases LDLR expression and density on the cell-surface, driving this feedback loop. Accurate measurement of PCSK9 concentration can be used to better understand patient response to statin treatment and whether PCSK9 therapy is likely to be more effective alone or with statin therapy. Shapiro et al., “Targeting PCSK9 for Therapeutic Gains,” Curr Artheroscler Rep 17(19): 1-9 (2015).

A 692 amino acid human variant of PCSK9 is described in NCBI protein database, accession number Q8NBP7 with the protein sequence of SEQ ID NO 1. An antibody may be directed toward a portion of this PCSK9 protein sequence or other variants, as appreciated by those skilled in the art. Anti-PCSK9 antibodies used in the disclosure may be polyclonal or monoclonal and may be directed specifically to a conserved region of the protein.

In an embodiment, the anti-PCSK9 antibody may be a mouse antibody. Non-limiting examples of commercially available PCSK9 antibodies include, ab125251 (Abcam, Cambridge, Mass., USA), MAB3888 (R&D Systems, Minneapolis, Minn., USA), 10008811 (Cayman Chemical, Ann Arbor, Mich., USA).

In an embodiment, one or more anti-PCSK9 antibodies may be directed toward a particular variant of PCSK9, such as described in U.S. Pat. No. 9,045,547, incorporated herein in its entirety, where a unique PCSK9 variant comprises an epitope with at least one of the following residues: S153, I154, P155, R194, D238, A239, I369, S372, D374, C375, T377, F379, V380, or S381 of SEQ ID NO 2.

In another embodiment, the one or more anti-PCSK9 antibodies may be directed toward a particular variant of PCSK9, described in U.S. Pat. No. 9,051,378, incorporated herein in its entirety, where a unique variant comprise an epitope on PCSK9 comprises a C-terminal domain comprising a mutation I474V or E670G in SEQ ID NO: 3.

Providing antibodies directed to unique PCSK9 variants individually, or with multiple unique antibodies and targets facilitates additional detection of PCSK9 variants of particular interest. Multivalent anti-sera can be produced with said antibodies as described further herein and as appreciated by one skilled in the art.

Lipo-IFE can be used to determine lipoprotein-bound PCSK9, free PCSK9, Apo B, and/or unbound lipoprotein their ratios by probing with anti-PCSK9 and anti-Apo B in a gel substrate which also produces a spatial separation between lipoprotein types, thereby offering more detailed assessment of disease-promoting or health-protective species. ELISA, and other immunological methods can be used to determine this ratio without distinction of particle species in the binding and detection step, but rather by targeting particle characteristics, such as bound Apo B to distinguish triglyceride-rich proteins (TRLs) from non-TRLs and subsequent detection of PCSK9, among other combinations, such as those that target Lp(a) characteristics specifically.

Clinically, knowing the ratio of PCSK9 bound particles to non-bound (e.g., free) particles facilitates measurement of modification of the PCSK9 generated by the liver. For example, with less PCSK9 from a baseline or average, more LDL receptors and the less LDL-P are expected in serum, characterizing reduced cardiovascular disease (CVD) risk. The concentrations of free- and bound-PCSK9 provide useful information about the overall CVD health of a subject.

There is a 1:1 relationship between PCSK9 and the particles to which they are bound. The PCSK9 bound to lipid particles have increased clinical significance relative to non-bound PCSK9, because from them information about activity, specificity, and affinity between PCSK9 and LDLR can be directly determined. Indirectly, additional information about structure and lifetime can be determined, all of which further indicate the ongoing health of a patient given their PCSK9 and LDLR chemical properties. Existing total serum methods, described herein, are not able to differentiate between bound and non-bound PCSK9 and therefore fail of offer these useful insights

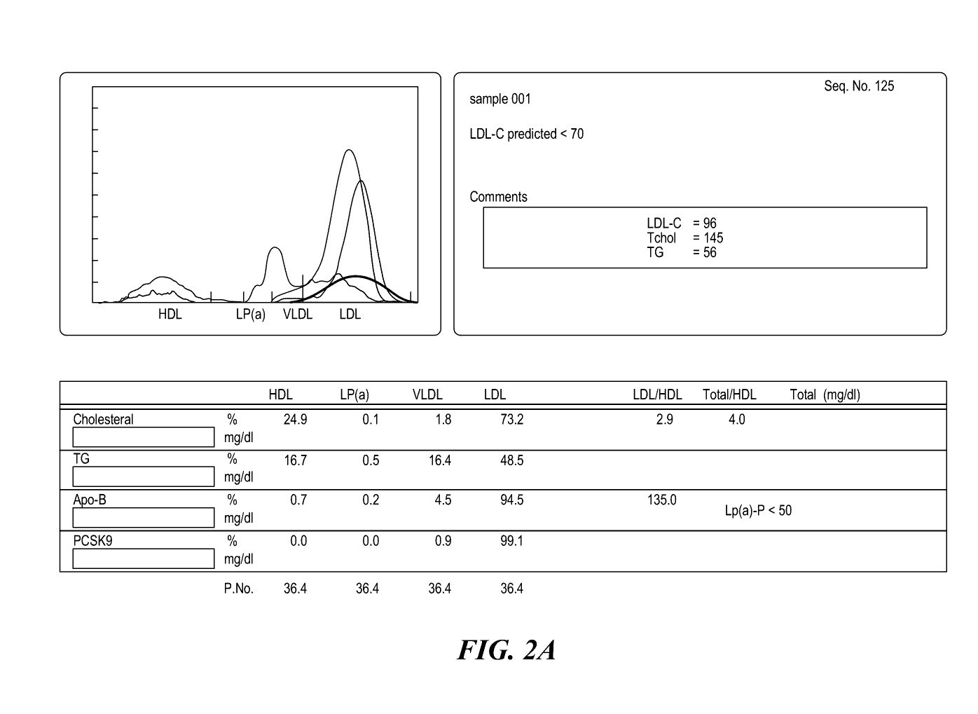

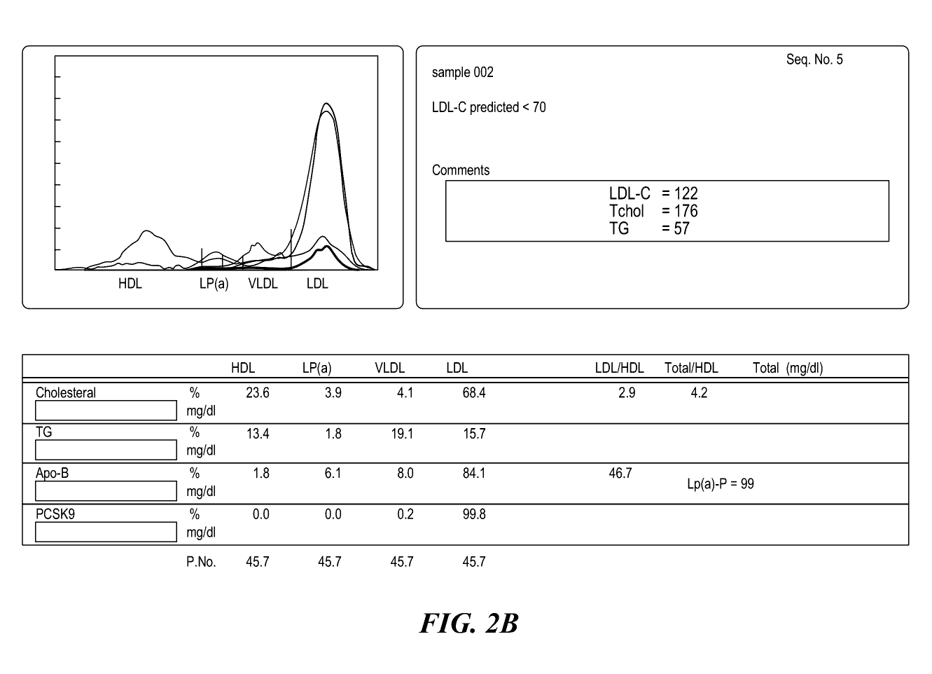

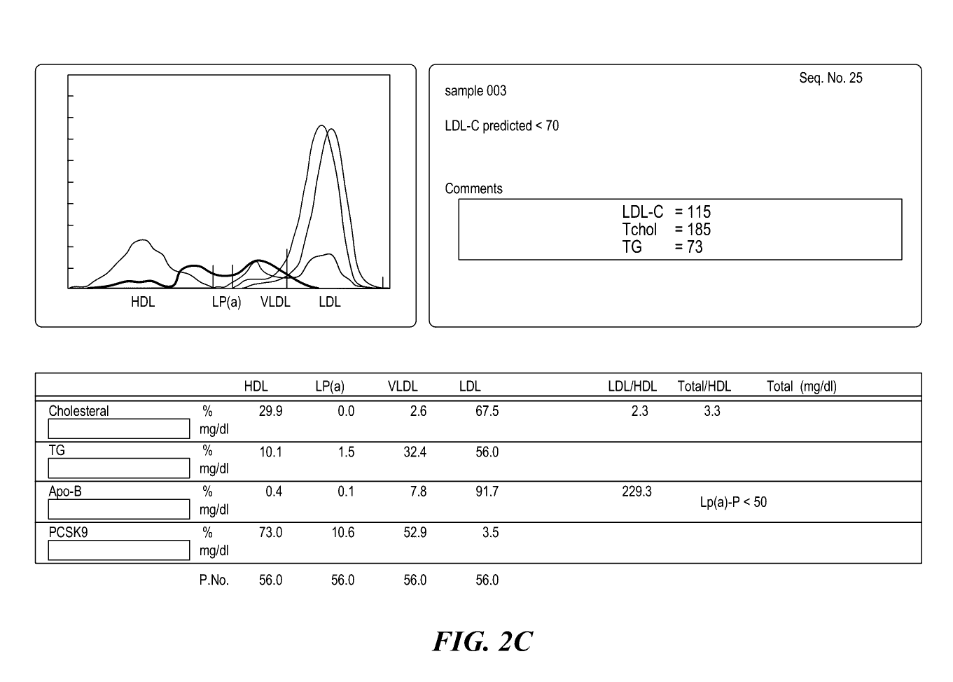

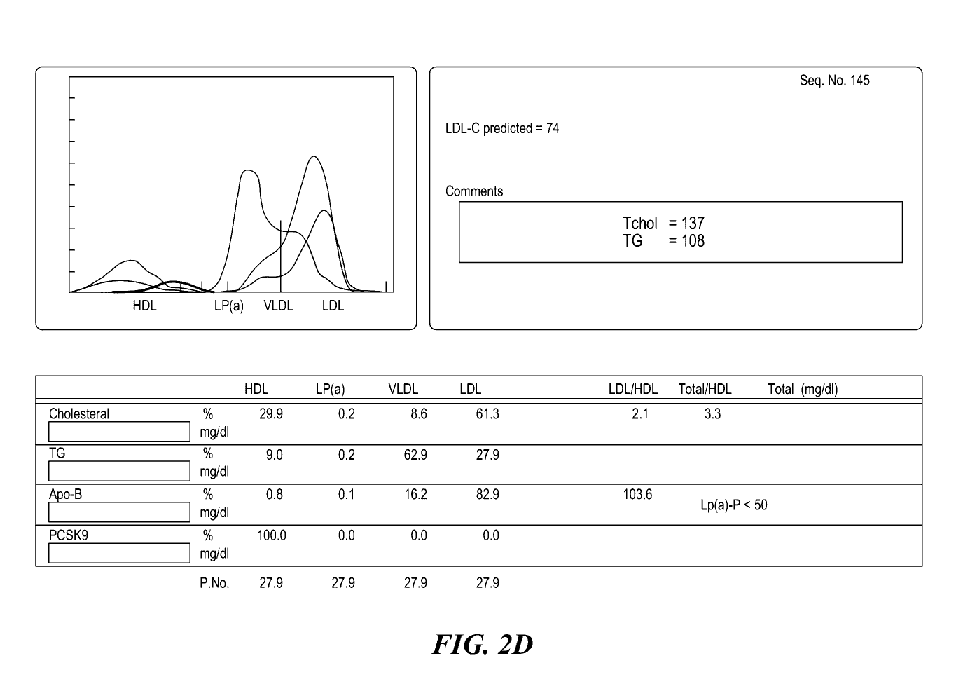

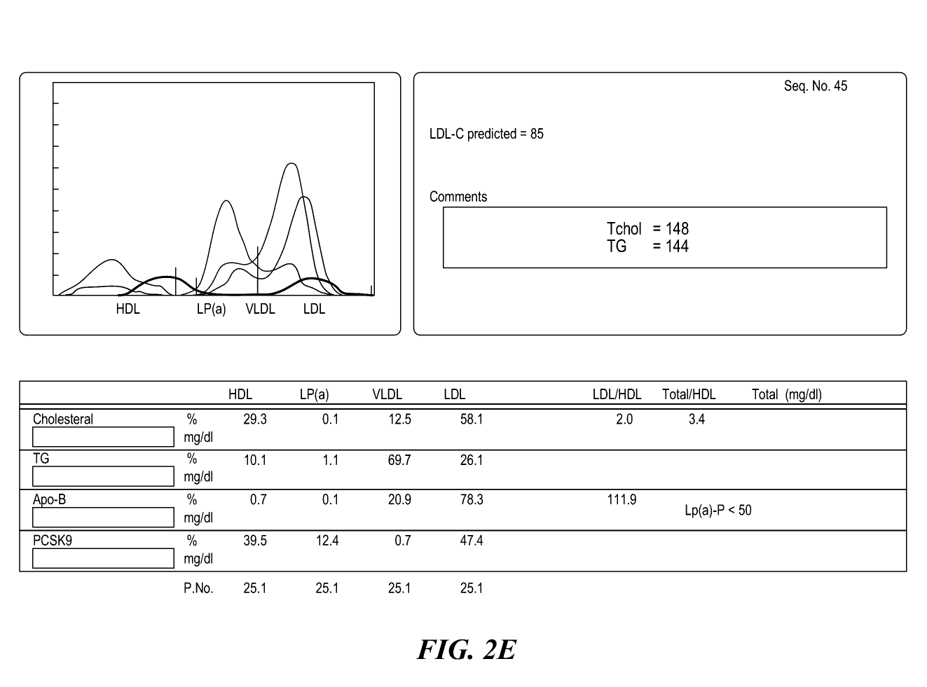

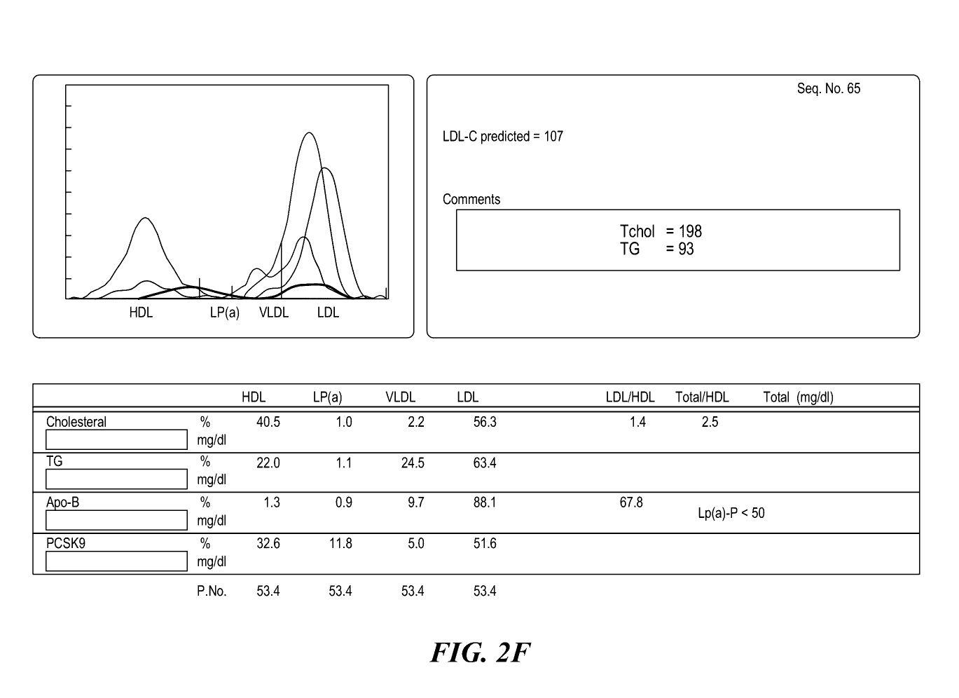

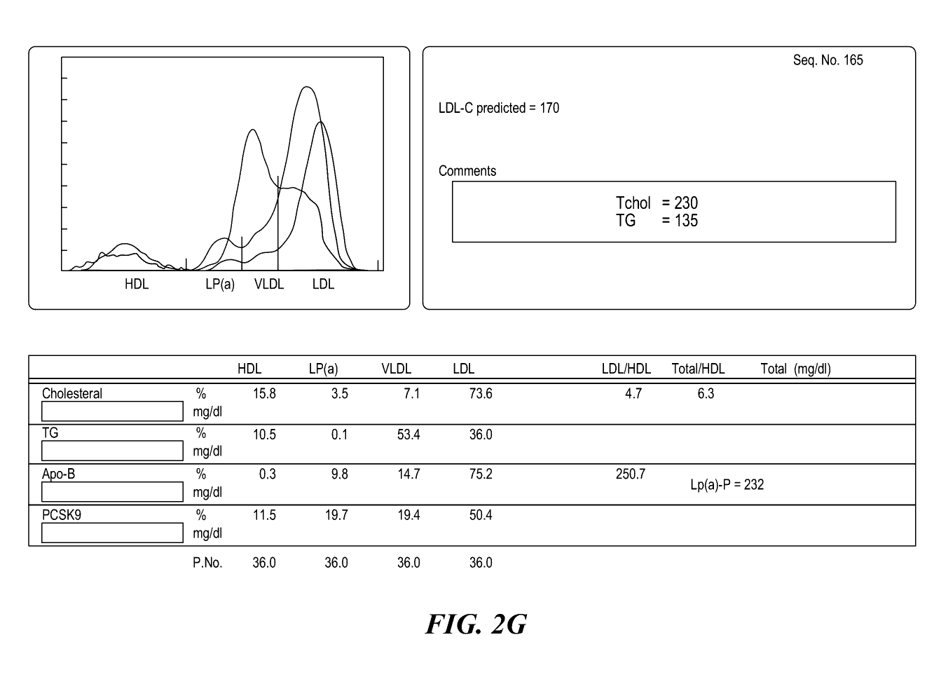

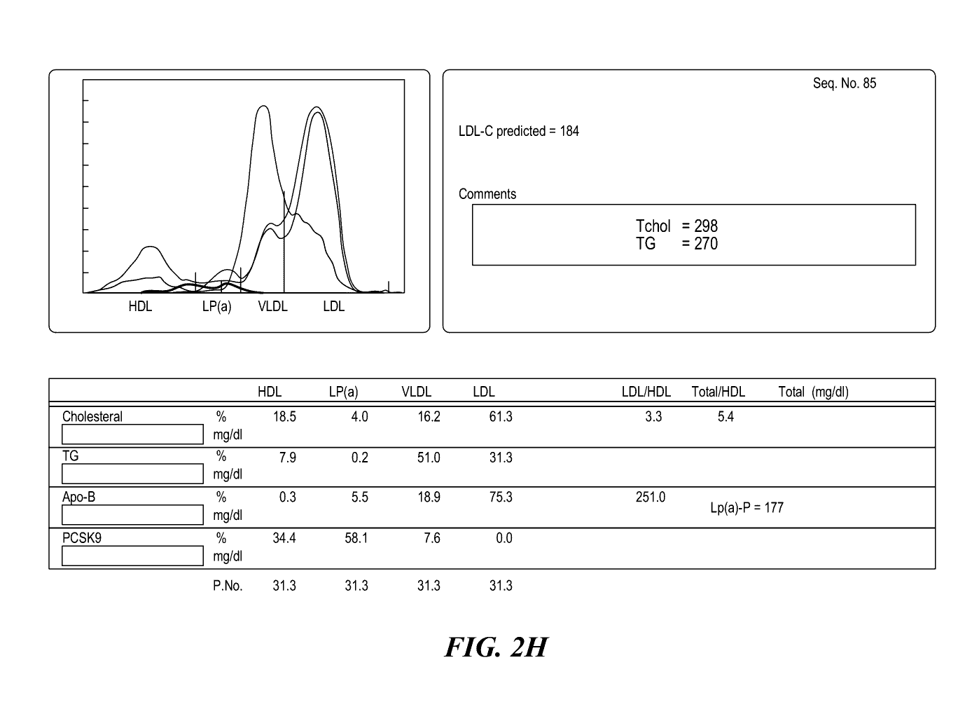

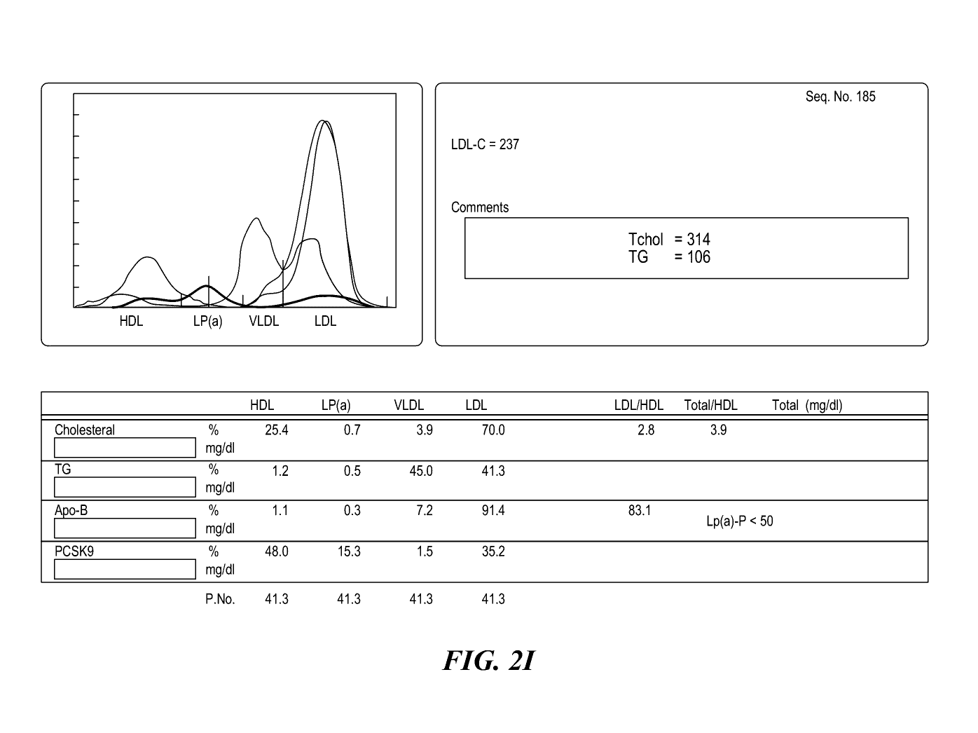

System and methods for the measurement of PCSK9 and Apo B can be adapted from the Lipo-IFE method presented in US Publication No. 20140243431A1 and U.S. Pat. No. 9,005,419. Current results, as presented in FIGS. 2A-H and described below, show that PCSK9 positions in the resolved gel substrate are consistent with lipid particle positions measured by Apo B detection. Although the sample solution contains lipid particle-bound and free PCSK9, the free PCSK9 have increased migration velocities relative to the lipid particle-bound PCSK9 and are beyond the electrophoretogram scanning window. The charge on a non-bound PCSK9 is different in charge and hydrodynamic resistance to that of a lipid particle with its associated lipoproteins and PCSK9.

Reagents incorporated into the system and method are adapted from Lipo-IFE methods with the substitution of anti-PCSK9 for anti-lipoprotein (e.g., anti-Apo B) as described in U.S. Pat. No. 9,005,419. This substitution will fix those particles bound with PCSK9 for subsequent staining.

In Lipoprotein Immunofixation Electrophoresis (Lipo-IFE), a system comprising a lipoprotein-containing sample, electrophoretic gel, antibody targeted to a particular lipoprotein component, optically-active component, and visualization system used in a separation-fixation-visualization protocol as described in US Publication No. 20120052594 and US Publication No. 20140243431, incorporated herein in their entirety, and as follows. The lipoprotein-containing sample is applied to a spot on a gel substrate suitable for zonal gel electrophoresis. In zonal gel electrophoresis, the lipoproteins are separated according to their isoelectric points in a non-denaturing gel that acts as a molecular sieve. In one embodiment, the gel may be an agarose gel. Velocity of a particle, and subsequently its final position after a voltage has been applied, is determined by a particle's ability to migrate through the gel matrix. The voltage generates movement in the particles by attracting or repulsing the total inherent charge of the particle surface relative to the solvent carrier and the other particles in solution. Charge of the particle is characterized by the number of ionized groups on the amino acid content of the proteins, which generate an overall charge to the particle that will respond uniquely to the voltage applied across the gel. This is distinguished from gradient gel electrophoresis, typically carried out in a polyacrylamide gel, which comprises a pH gradient along the gel and in which the particles are “reduced,” meaning a reducing agent breaks bonds between apolipoproteins and the lipid particles, thereby separating these components which no longer comprise a “lipoprotein.” Reduction destroys information about the apolipoprotein content and removing an antigenic target for detection from the lipid particle. Zonal gel electrophoresis is unique in its ability to preserve overall structure and therefore the information inherent in that structure. The native structure information is more useful to assessment of a patient's condition than population totals of Apo B and lipid particles, which are distinct conditions of matter than those found in a patient's body.

Anti-sera containing antibodies targeted to a particular portion of one or more lipoproteins, such as an apolipoprotein on the lipoprotein surface, is applied to the gel substrate over the separated lipoproteins. After some time for incubation, the excess antisera is washed from the gel. The antibodies have by now bound to the lipoproteins, preventing their further movement within the gel substrate, so washing removes the unbound solutes from the gel. If the antisera is monovalent, meaning it targets only one antigen, a nonspecific protein dye may be used to dye the bound antibodies. After further incubation and washing, the density of dye at spots on the gel is measured by a means of optical detection such as a densitometer. A qualitative measurement (presence or absence) of a target component can be detected. If a control sample is also incubated with dye and measured, it may be possible to quantify the level of the antigen component and calculate a particle number.

As noted above, an antibody complex may include an antibody that binds a lipoprotein particle or a portion thereof, where the antibody is bound to a signal producing molecule capable of producing or causing production of a detectable signal.

Calibration of the assay requires an internal standard with signal-production means of equivalent function and concentration as the antibodies targeting PCSK9 or Apo B. In an embodiment, a particle or protein such as albumin, with known concentration, is labeled with the same fluorescent label as the antibody targeting PCSK9 or Apo B. The labeled albumin, having a known gel migration velocity that is distinct from the PCSK9 or Apo B bound particle, can be measured by the optical detection mechanism, such as a fluorimeter, and its signal/concentration ratio used to calculate the concentration of PCSK9 or Apo B antibodies, corresponding to the related particle number, through calculations known in the art. The 1:1 particle:protein ratios for both PCSK9 and Apo B facilitate the accurate quantitation of underlying particle number.

In a preferred embodiment, the antisera contains anti-Apo B antibodies, which can be used to quantify triglyceride rich lipoproteins such as LDL, VLDL, Lp(a), or IDL components. The antisera may also contain anti-apo(a) components to target Lp(a) components specifically. In the current invention, anti-PCSK9 antibodies are included in the antisera to specifically bind and label PCSK9 components on a lipoprotein. The function of the antibodies in Lipo-IFE is both to fix the lipoproteins in the gel substrate matrix and provide an indirect means for detecting the presence of the antigen target. Antibodies directed to the PCSK9 protein will not fix free PCSK9 in the gel substrate, because the resultant antibody-PCSK9 complex is too small to be bound in the gel and will escape in a washing step, leaving only lipoprotein-bound level of PCSK9, and optionally free PCSK9, Apo B and/or PCSK9 unbound lipoproteins for detection.

In a preferred embodiment, the antibodies used to target lipoprotein components are bound to a fluorescent label for detection by means other than non-specific dye densitometry. A fluorescent label-bound antibody provides the ability to include multivalent antisera, targeting multiple antigens in the same spot and measuring their presence or amount through fluorimetry so long as the fluorescent labels targeting different antigens are optically distinct, such as by fluorescing at unique wavelengths. Furthermore, a calibration and standardization method may be used to facilitate quantification, as described in U.S. Pat. No. 9,005,419, incorporated herein in its entirety.

Enzyme-linked immunosorbent assays (ELISAs) are an alternative immunological assay to the electrophoretic assay that do not rely on lipoprotein separation in a gel substrate. ELISAs are well-known in the art, and may involve either a sandwich assay, competitive assay, or direct antigen detection in this application.

In a direct method, a capture antibody, targeted to some antigenic portion of a particle of interest, such as the Apo B portion of a triglyceride-rich lipoproteins, is bound to a solid substrate and captures the particles as they are incubated with the bound antibodies or as the antibodies bound to small inert particles are mixed into a solution of Apo B-containing lipoproteins. The solid substrate may be the surface of a plate, a bead, a test tube or other solid portion to which the antibody can be bound. Another detection antibody, directed to some portion of the same particle, for instance, the PCSK9 portion of an LDL, is also mixed into solution or incubated with the bound particle. The detection antibody has some means of being detected or producing a signal, such as through fluorescence or radioactivity or other means widely documented and described in the art. Presence and absence and levels of antigenic target for the detection assay can be measured in this manner.

ELISA methods are more extensively described in US Publication No. 20150168427, incorporated herein in its entirety. The advantage of the ELISA assay over alternatives, such as the electrophoretic assay, is higher efficiency and lower cost per sample. This is an attractive feature for high-throughput reference laboratories, where thousands of tests are run in a day, and incremental costs can be limited through automation and fewer reagents or materials. While providing a more limited type of information about the sample, the efficiency is beneficial.

In a more general sandwich assay, a surface is prepared to which a known quantity of capture antibody is bound. Any nonspecific binding sites on the surface are blocked. The antigen-containing sample is applied to the plate, and captured by the antibody. The plate is washed to remove unbound antigen. A specific antibody is added and binds to antigen. This primary antibody could also be in the serum of a donor to be tested for reactivity towards the antigen. Enzyme-linked secondary antibodies are applied as detection antibodies that also bind specifically to the antibody's Fc region. The plate is washed to remove the unbound antibody-enzyme conjugates. A chemical is added to be converted by the enzyme into a color or fluorescent or electrochemical signal. The absorbance or fluorescence or electrochemical signal of the plate wells is measured to determine the presence and quantity of antigen.

In an indirect sandwich assay, a capture antibody, targeted to some antigenic portion of a particle of interest, such as the Apo B portion of a triglyceride-rich lipoproteins, is bound to a solid substrate and captures the particles as they are incubated with the bound antibodies or as the antibodies bound to small inert particles are mixed into a solution of Apo B-containing lipoproteins. The solid substrate may be the surface of a plate, a bead, a test tube or other solid portion to which the antibody can be bound. A primary detection antibody, directed to some portion of the same particle, for instance, the PCSK9 portion of an LDL, is also mixed into solution or incubated with the bound particle. Finally, a secondary antibody directed to the detection antibody and having some means of being detected or producing a signal such as through fluorescence or radioactivity is incubated with the primary detection antibody, labeling bound primary detection antibodies and facilitating measurement through optical or other signal-producing means. Presence and absence and levels of antigenic target for the detection assay can be measured in this manner.

Additional immunochemical techniques may be applied to the measurement and detection of lipoprotein-bound PCSK9 and/or Apo B, including, without limitation, immunoturbidometric assays, other agglutination methods that rely on antibody binding and agglutination of microparticles (the solid substrates), light scattering immunoassays, lateral flow systems, immunofluorescence methods, immunostaining methods, chemiluminescence methods and precipitin tests, all described in Koivunen et al. “Principles of Immunochemical Techniques Used in Clinical Laboratories” Lab Medicine 37:8 (2006), incorporated herein in its entirety.

As used herein, the term “antibody” is meant to include intact immunoglobulins derived from natural sources or from recombinant sources, as well as immunoreactive portions (i.e. antigen binding portions) of intact immunoglobulins. The antibodies of the invention may exist in a variety of forms including, for example, polyclonal antibodies, monoclonal antibodies, intracellular antibodies, antibody fragments (e.g., Fv, Fab and F(ab)2), as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc Natl Acad Sci USA 85:5879-5883 (1988); Bird et al, “Single-Chain Antigen-Binding Proteins,” Science 242:423-426 (1988), which are hereby incorporated by reference in their entirety). In some embodiments, the anti-lipoprotein antibody is selected from the group consisting of anti-lipoprotein (a), anti-apolipoprotein B (Apo B), and anti-apolipoprotein-a (apo-a). In one preferred embodiment, the anti-lipoprotein antibody is Apo B. In some embodiments, the antibody can be a biosimilar.

Methods for monoclonal antibody production may be carried out using techniques well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Procedures for raising polyclonal antibodies are also well known (Ed Harlow and David Lane, USING ANTIBODIES: A LABORATORY MANUAL (Cold Spring Harbor Laboratory Press, 1988), which is hereby incorporated by reference in its entirety).

For example, polyclonal antibodies to an apolipoprotein may be produced by injecting a suitable animal host, such as a rabbit, with the apolipoprotein of interest and an adjuvant. Approximately 0.02 milliliters may be injected, with reinjection occurring every 21 days until peak antibody titer is achieved. Antibody titer may be tested by, for example, an ear bleed. Antibodies to PCSK9, Apo B-100 or other apolipoprotein may be produced in this manner. Alternatively, antibodies to PCSK9, Apo B-100 or other apolipoprotein may be purchased commercially.

In addition to whole antibodies, the invention encompasses binding portions of such antibodies. Such binding portions include the monovalent Fab fragments, Fv fragments (e.g., single-chain antibody, scFv), single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES: PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

Suitable signal producing molecules that are capable of producing or causing production of a detectable signal will be known to those of skill in the art. The detectable signal includes any signal suitable for detection and/or measurement by radiometric, colorimetric, fluorometric, size-separation, or precipitation means, or other means known in the art.

Examples of signal producing molecules that are capable of producing or causing production of a detectable signal include various enzymes, prosthetic groups, fluorescent materials, luminescent materials, bioluminescent materials, radioactive materials, positron emitting metals, and nonradioactive paramagnetic metal ions. The signal producing molecules may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the invention. Further examples include, but not limited to, various enzymes. Examples of enzymes include, but are not limited to, horseradish peroxidase, alkaline phosphatase, beta-galactosidase, or acetylcholinesterase; prosthetic group complexes such as, but not limited to, streptavidin/biotin and avidin/biotin. Examples of fluorescent materials include, but are not limited to, umbelliferone, fluorescein, fluorescein isothiocyanate, rhodamine, dichlorotriazinylamine fluorescein, dansyl chloride or phycoerythrin. Examples of luminescent material include, but are not limited to, luminol. Examples of bioluminescent materials include, but not limited to, luciferase, luciferin, and aequorin. Examples of radioactive material include, but are not limited to, bismuth (213Bi), carbon (14C), chromium (51Cr), (153Gd, 159Gd)5 gallium (68Ga, 67Ga), germanium (68Ge), holmium (166Ho), indium (115In, 113In, 112In, 111In), iodine (1311, 1251, 1231, 1211), lanthanium (140La), lutetium (177Lu), manganese (54Mn), molybdenum (99Mo), palladium (103Pd), phosphorous (32P), praseodymium (142Pr), promethium (149Pm), rhenium (186Re, 188Re), rhodium (105Rh), ruthemium (97Ru), samarium (153Sm), scandium (47Sc), selenium (75Se), strontium (85Sr), sulfur (35S), technetium (99Tc), thallium (201Ti), tin (113Sn, 117Sn), tritium (3H), xenon (133Xe), ytterbium (169Yb, 175Yb), yttrium (90Y), zinc (65Zn). Further examples include positron emitting metals using various positron emission tomographies, and nonradioactive paramagnetic metal ions.

Detection of an antibody-signal producing molecule complex in accordance with the invention may also be achieved by addition of a reagent capable of interacting with the signal producing molecule, where the signal producing molecule produces a detectable signal upon contact with the reagent. For example, light is emitted when luciferase acts on the appropriate luciferin substrate.

A secondary antibody that is coupled to a detectable signal or moiety, such as for example, an enzyme (e.g., luciferase), fluorophore, or chromophore may also be used.

As noted above, each detectable signal of the at least two lipoprotein-binding complexes is distinguishable from the other detectable signal. This permits cocktailing at least two lipoprotein-binding complexes where each of the complexes detects a different lipoprotein particle or a portion thereof, each complex also producing or capable of producing a different detectable signal. For example, a first lipoprotein-binding complex may include fluorescein isothiocyanate (FITC)-labeled anti-Apo A1 antibody and a second lipoprotein-binding complex may include rhodamine-labeled anti-Apo A2 antibody. As another example, a first lipoprotein-binding complex may include fluorescein isothiocyanate (FITC)-labeled anti-Apo B antibody and a second lipoprotein-binding complex may include rhodamine-labeled anti-PCSK9 antibody. The first and second complexes may be mixed or cocktailed together and/or with additional (e.g., third, fourth, fifth, sixth, etc) complexes including antibodies that may recognize different lipoprotein particles or a portions thereof bound to further distinguishable signal-producing molecules. This permits probing of multiple antigens in a single electrophoretic lane.

For example, the signal producing molecules may include fluorescent tags. Fluorescence tagging and the detection of natural fluorescence in molecules is a method of analytical chemistry and biology that is well known in the art. The instruments used to detect fluorescence may include the following components. A light source with a broad optical bandwidth such as a light bulb or a laser is used as the source of the stimulating light. An optical filter is used to select the light at the desired stimulation wavelength and beam it onto the sample. Optical filters are available at essentially any wavelength and are typically constructed by the deposition of layers of thin film at a fraction of the wavelength of the desired transmission wavelength. The light that exits the optical filter is then applied to the sample to stimulate the fluorescent molecule.

The molecule then emits light at its characteristic fluorescent wavelength. This light is collected by a suitable lens and is then passed through a second optical filter centered at the characteristic wavelength before being brought to a detection device such as a photomultiplier tube, a photoconductive cell, or a semiconductor optical detector. Therefore, only light at the desired characteristic wavelength is detected to determine the presence of the fluorescent molecule. Accordingly, the at least two lipoprotein-binding complexes may include fluorescent molecules that emit light at different, distinguishable fluorescent wavelengths.

Fluorescent tags may be multiplexed in a single area such that they are optically distinct. For example, 5 different fluorescent tags, red, green, blue, yellow, and orange may be applied to the same limited area and be independently detected and distinguished by optical detection software. For example, the Alexa Fluor® product line includes at least 19 distinct dyes that may be combined for tagging distinct antibodies to label and identify individual antigens (ThermoFisher Scientific, Waltham, Mass., USA).

An optical system can quantitate the fluorescent signals and automatically normalize the signal value to generate relative densities or particle numbers. For example, by normalizing the extinction/emission coefficients or quantum relativity of each dye, relative values for concentration or particle number can be determined.

The system and methods may also include a device or use of a device for detecting the detectable signal, where the detecting indicates the level of the specific lipoprotein-bound PCSK9 and/or Apo B proteins in the biological sample. The device may also quantitate the level of specific lipoprotein-bound PCSK9 and/or Apo B based on the detection of the signal producing molecule.

The presence of the lipoprotein-bound PCSK9 and/or Apo B in the electrophoretic gel may be quantified by measurement of the detectable signal or moiety. A particle number may then be calculated according to known stoichiometric relationships as there is a 1:1 stoichiometry of Apo B to LDL particles and PCSK9 to LDL particles. The 1:1 stoichiometry may be exploited to quantify other properties of the LDL-containing sample, such as the Apo B:PCSK9 ratio and the LDL:PCSK9 ratio. The particle number may be quantified by comparison with a separate analysis that characterizes the total lipid particle or class of lipid particle concentration in the sample. Such separate analysis may be ultracentrifugation, NMR, or any other analysis method that can characterize a concentration or total particle number for particles in the sample. Said sample used in lipid particle electrophoresis and lipid particle quantification may be different aliquots of the same sample.

In some embodiments, total PCSK9 (bound and unbound) is measured. PCSK9 is found in plasma primarily in two forms: (1) a 62-kDa protein representing the full-length (i.e., intact) protein, minus the pro-domain and (2) a 55-kDa fragment produced by protease furin. In some embodiments, the PCSK9 measured is the mature form, the furin-cleaved form, or both. In some embodiments, the mature form is expressly excluded. In other embodiments, the furin-cleaved form is expressly excluded. In some embodiments, elevated PCSK9 is in the absence of elevated LDL cholesterol.

One aspect of the disclosure relates to a method for measuring improvement or worsening of a patient undergoing treatment for hypercholesterolemia, said method comprising: obtaining a biological sample from the patient; measuring the amount of lipoprotein-bound PCSK9, free PCSK9, or both in the biological sample; measuring the amount of lipoprotein-bound Apo B, free Apo B, or both in the biological sample; optionally measuring an amount of triglyceride-rich lipoprotein (TRL) in the biological sample; generating a ratio of bound:free PCSK9, of bound PCSK9:Apo B, and/or of bound PCSK9:TRL; and comparing the ratio(s) generated to a reference level(s).

In some embodiments, the methods for measuring improvement or worsening of a patient undergoing treatment for hypercholesterolemia further comprise evaluating the level of improvement or worsening in hypercholesterolemia of the patient by a decreasing or increasing ratio.

In some embodiments, worsening of the patient undergoing treatment for hypercholesterolemia is determined when there is a greater level of PCSK9 than the reference level, the ratio of bound:free PCSK9 is greater than the reference level, the ratio of bound PCSK9:Apo B is greater than the reference level, or the ratio of bound PCSK9:TRLs is greater than the reference level. The ratio may be about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or greater than the reference level.

In some embodiments, improvement of the patient undergoing treatment for hypercholesterolemia is determined when there is a lower level of PCSK9 than the reference level, the ratio of bound:free PCSK9 is less than the reference level, the ratio of bound PCSK9:Apo B is less than the reference level, or the ratio of bound PCSK9:TRLs is greater than the reference level. The ratio may be about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or less than the reference level.

In some embodiments, the reference value is a level of the lipoprotein-bound PCSK9, free PCSK9, lipoprotein-bound Apo B, free Apo B, triglyceride-rich lipoprotein, or any ratio thereof determined from a sample collected from a healthy patient not having hypercholesterolemia. In other embodiments, the reference value is a level of the lipoprotein-bound PCSK9, free PCSK9, lipoprotein-bound Apo B, free Apo B, triglyceride-rich lipoprotein, or any ratio thereof determined from a plurality of samples collected from a population of healthy patients not having hypercholesterolemia. In other embodiments, the reference value is a normalized level of the lipoprotein-bound PCSK9, free PCSK9, lipoprotein-bound Apo B, free Apo B, triglyceride-rich lipoprotein, or any ratio thereof determined from a sample collected from a healthy patient not having hypercholesterolemia. In yet other embodiments, the reference value is a level of the lipoprotein-bound PCSK9, free PCSK9, lipoprotein-bound Apo B, free Apo B, triglyceride-rich lipoprotein, or any ratio thereof determined from a sample collected at an earlier time point (e.g., 1 day, 3 days, 1 week, 1 month, 3 months, 6 months, 12 months, or more) from the same patient which is undergoing treatment for hypercholesterolemia. In some embodiments, the reference value may be based on values known by those of skill in the art or developed by a medical agency.

In some embodiments, these detection methods are used in combination with traditional cardiovascular risk factors to determining CVD risk assessment. In some embodiments, PCSK9 levels are used as an independent CVD risk factor.

A further aspect of the invention relates to a method of determining whether a subject is at increased risk for cardiovascular disease. One aspect relates to methods for assessing risk of a patient developing cardiovascular disease or progression of cardiovascular disease, said method comprising: obtaining a biological sample from the patient; measuring an amount of lipoprotein-bound PCSK9, free PCSK9, or both in the biological sample; measuring an amount of lipoprotein-bound Apo B, free Apo B, or both in the biological sample; optionally measuring an amount of triglyceride-rich lipoprotein (TRL) in the biological sample; generating a ratio of bound:free PCSK9, of bound PCSK9:Apo B, and/or of bound PCSK9:TRL; and comparing the ratio(s) to a reference level(s).

In some embodiments the methods further comprise, evaluating the level of risk of developing or progression of cardiovascular disease by a decreasing or increasing ratio.

These methods include assessing the level of specific PCSK9 and/or apolipoproteins and/or lipoprotein particles present in a biological sample. The assessing includes the steps of providing a biological sample comprising lipoprotein particles and providing at least two lipoprotein-binding complexes. Each complex includes an antibody that binds a lipoprotein particle or a portion thereof, where the antibody is bound to a signal producing molecule capable of producing or causing production of a detectable signal. Each detectable signal of separate protein-binding complexes (PCSK9 or Apo B) is distinguishable from the other detectable signal. The assessing step also includes separating the lipoprotein particles present in the biological sample by depositing the biological sample on an electrophoretic gel and carrying out gel electrophoresis; contacting the biological sample with protein-binding complexes under conditions suitable to form a lipoprotein-antibody-signal producing molecule complex; washing the gel to eliminate, or substantially eliminate, unbound antibody; detecting the detectable signal produced by the signal producing molecule of the lipoprotein-antibody signal producing molecule complex on the electrophoretic gel; and determining the level of the specific lipoprotein-bound PCSK9 and/or Apo B present in the biological sample based on the detecting. The method also includes the step of correlating the determined level of the specific lipoprotein-bound PCSK9 and/or Apo B to a control or reference value to determine if the subject is at an increased risk for cardiovascular disease.

The assessment may include separating lipoprotein particles present in the biological sample by depositing the biological sample on an electrophoretic gel and carrying out gel electrophoresis; forming two or more PCSK9 or Apo B protein-antibody-signal producing molecule complexes, where the one or more antibodies are specific to one or more different proteins or a portion thereof; detecting the detectable signal produced by the signal producing molecules of the respective protein-antibody-signal producing molecule complexes on the electrophoretic gel; and determining the levels of the different PCSK9 and/or apolipoproteins and lipoprotein particles present in the biological sample based on the detecting.

Correlation in the context of lipid-related health risk, cardiovascular condition, and risk of cardiovascular disease, refers to a statistical correlation of the resulting distribution of lipoprotein-bound and unbound PCSK9 with population mortality and risk factors, as well other factors known in the art. Correlation in the context of monitoring cardiovascular risk (e.g., for responsiveness to a therapeutic intervention) refers to comparison of the distribution of lipoprotein-bound and unbound PCSK9 at two time points (e.g., before and after a therapeutic intervention is conducted).

The correlating may include correlating the determined levels of the different lipoprotein-bound PCSK9 and/or Apo B to a control or reference value to determine if the subject is at an increased risk for cardiovascular disease. In some embodiments, the reference value is a level of the lipoprotein-bound PCSK9, free PCSK9, lipoprotein-bound Apo B, free Apo B, triglyceride-rich lipoprotein, or any ratio thereof determined from a sample collected from a healthy patient not having cardiovascular disease. In other embodiments, the reference value is a level of the lipoprotein-bound PCSK9, free PCSK9, lipoprotein-bound Apo B, free Apo B, triglyceride-rich lipoprotein, or any ratio thereof determined from a plurality of samples collected from a population of healthy patients not having cardiovascular disease. In other embodiments, the reference value is a normalized level of the lipoprotein-bound PCSK9, free PCSK9, lipoprotein-bound Apo B, free Apo B, triglyceride-rich lipoprotein, or any ratio thereof determined from a sample collected from a healthy patient not having cardiovascular disease. In yet other embodiments, the reference value is a level of the lipoprotein-bound PCSK9, free PCSK9, lipoprotein-bound Apo B, free Apo B, triglyceride-rich lipoprotein, or any ratio thereof determined from a sample collected at an earlier time point (e.g., 1 day, 3 days, 1 week, 1 month, 3 months, 6 months, 12 months, or more) from the same patient for which risk is being assessed. In some embodiments, the reference value may be based on values known by those of skill in the art or developed by a medical agency.

In some embodiments, an increased risk for cardiovascular disease is determined when there is a greater level of PCSK9 than the reference level, the ratio of bound:free PCSK9 is greater than the reference level, the ratio of bound PCSK9:Apo B is greater than the reference level, or the ratio of bound PCSK9:TRLs is greater than the reference level. The ratio may be about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or greater than the reference level.

In some embodiments, a decreased risk for cardiovascular disease is determined when there is a lower level of PCSK9 than the reference level, the ratio of bound:free PCSK9 is less than the reference level, the ratio of bound PCSK9:Apo B is less than the reference level, or the ratio of bound PCSK9:TRLs is greater than the reference level. The ratio may be about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or less than the reference level.

The correlating may also include assigning the subject to a risk category selected from the group consisting of high risk, intermediate risk, and low risk (or optimal) groups for developing or having cardiovascular disease. There are well established recommendations for cut-off values for biochemical markers (for example, and without limitation, cholesterol and lipoprotein levels) for determining risk. There are guidelines for recommended cut-off values of Apo B for determining risk. Furthermore, careful statistical development of robust PCSK9 cut-offs, accounting for the distribution on- and off-lipoprotein particles, by demographic characterization, and in combination with other risk factors are a part of the current invention.

The invention also includes selecting a therapy regimen based on the risk for cardiovascular disease determined. For instance, an individual may be determined to be at an elevated risk according to the methods and a treatment regimen may then be selected based on the elevated risk.

The selected therapy regimen may include drugs or supplements. Suitable drugs or supplements include those administered for the purpose of modifying PCSK9 concentration or activity, lowering serum cholesterol, lowering LDL, IDL, and VLDL, Lp(a) and/or raising HDL, as known in the art.

Examples of suitable drugs include a PCSK9 inhibitors, anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, or combinations thereof.

The selected therapy regimen may also involve giving recommendations on making or maintaining lifestyle choices based on the risk for cardiovascular disease determined. Lifestyle choices may involve changes in diet, changes in exercise, reducing or eliminating smoking, or a combination thereof.

A report may also be generated that includes, among other things, a description of the selected treatment regimen. In some embodiments, the results of lipoprotein analyses (lipoprotein-bound and unbound PCSK9) are reported in such a report. A report refers in the context of lipoprotein and other lipid analyses to a report provided, for example to a patient, a clinician, other health care provider, epidemiologist, and the like, which includes the results of analysis of a biological specimen, for example a plasma specimen, from an individual. Reports can be presented in printed or electronic form, or in any form convenient for analysis, review and/or archiving of the data therein, as known in the art. A report may include identifying information about the individual subject of the report, including without limitation name, address, gender, identification information (e.g., social security number, insurance numbers), and the like. A report may include biochemical characterization of the lipids in the sample, for example without limitation triglycerides, total cholesterol, LDL cholesterol, and/or HDL cholesterol, and the like. A report may further include characterization of lipoproteins, and reference ranges therefore, conducted on samples prepared by the methods provided herein. The term “reference range” and like terms refer to concentrations of components of biological samples known in the art to reflect typical normal observed ranges in a population of individuals. Exemplary characterization of lipoproteins in an analysis report may include the concentration and reference range for PCSK9, apolipoprotein AI, AII, AII, B, C, and/or E, and/or VLDL, IDL, Lp(a), LDL and HDL, and subclasses thereof. A report may further include lipoprotein size distribution trends.

The invention also may further include administering the selected treatment regimen to the subject. Accordingly, a further aspect of the present invention relates to a method of treating a subject having an elevated risk for cardiovascular disease determined.

The invention also relates to a method of monitoring the risk for developing cardiovascular disease. This method includes determining whether a subject is at increased risk for cardiovascular disease at a first time point and repeating the determining at one or more later time points (e.g., before and after therapeutic intervention or at progressive time points during a course of therapeutic intervention). The determined risk at each progressive time point is compared the determined risk from one or more earlier time points to evaluate whether the subject's risk for developing cardiovascular disease has increased or decreased, thereby monitoring the risk for developing cardiovascular disease. This method may involve assigning a risk category based on the determined risk for developing cardiovascular disease and comparing the risk categories assigned at progressive time points (e.g., comparing a first risk category determined at a first time point to a second risk category taken at a second time point), thereby monitoring the risk for developing cardiovascular disease.

As noted above, the prior electrophoretic technology required transfer protocols or traditional IFE non-protein specific staining of cross-linked probed complex. Further, existing strategies to measure detect and quantify lipid protein particles use nonspecific protein dye to detect the fixed proteins in a gel (U.S. 20120052594, which is hereby incorporated by reference in its entirety). This invention provides more efficient systems and methods to detect, quantify, and characterize PCSK9, apolipoproteins and lipoproteins particles that avoid the use of transfer-blot technology.

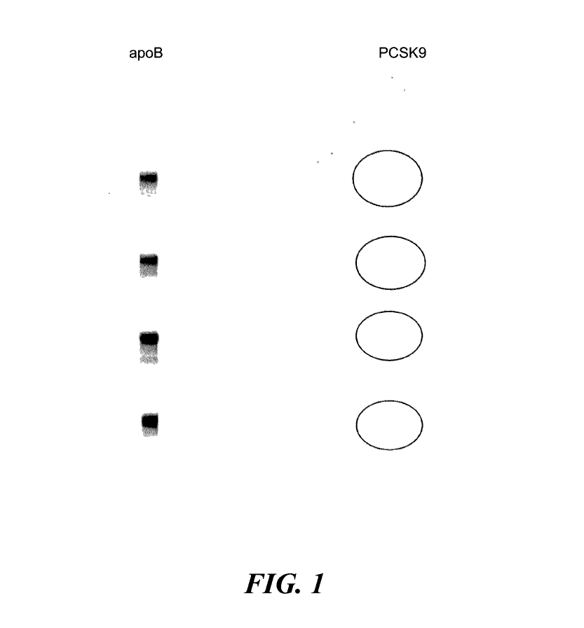

An example of a laboratory method of non-specific Lipo-IFE method is described and shown in FIG. 1. Generally, the protocol involves: 1) obtaining a biosample, 2) preparing the biosample to just include serum, 3) applying the biosample to the gel, 4) separating lipid particles by electrophoresis, 5) applying polyclonal antibodies to the gel to fix PCSK9, 6) washing the gel to get rid of anything that is not a lipid particle bound to an Antibody-PCSK9 complex, 7) staining the gel with a non-specific protein stain, 9) repeating the process in parallel for various immunologically-active targets such as Apo B in distinct lanes.

For each lane, antisera targeting PCSK9 or a distinct apolipoprotein species on a lipoprotein particle is applied to the gel fixing the lipoprotein/lipid particle complex in place in the lane and any associating additional protein with the particle. The gel is washed to remove non-immunofixed materials and a non-specific protein stain is applied to the gel. Subsequent analysis can be done to report relative amounts of lipoproteins in a lane, all of which are associated with the same apolipoprotein. The process is repeated in additional lanes with antisera to different apolipoproteins but using the same non-specific protein stain. Immunofixation and staining are carried out on the same gel as the electrophoresis procedure.

Antisera is composed of monoclonal or polyclonal antibodies targeted at a specific apolipoprotein as it exists on the surface of a lipoprotein particle. Because subsequent analysis is non-specific, the antisera can only be directed to a single antigen. Analysis is limited to relative levels of particles having the same apolipoprotein attached to the protein. An example of a method of analysis of a non-specific Lipo-IFE includes: 1) differentiating the detected bands in a gel lane by manually or automatically determining boundaries around each band, 2) associating each band with distinct lipoprotein-bound PCSK9, 3) assigning the bands to different particles, 4) obtaining a total concentration number for lipoprotein-bound PCSK9 from a different assay such as internal calibration on the gel, ultracentrifugation or NMR, 5) calculating the particle numbers based on optical density and position in the gel and absolute particle number, 6) assessing patient risk level for cardiovascular disease, diabetes mellitus, or any other condition by absolute reported particle level, and/or 7) calculating a ratio of particles levels with clinical significance and assessing patient risk level for cardiovascular disease, diabetes mellitus, or any other condition by particle level ratios, and 8) reporting said risk level.

On the other hand, an example of a method of the invention using specific Lipo-IFE includes: 1) obtaining a biosample, 2) preparing the biosample to just include serum, 3) applying the biosample to the gel, 4) separating lipid-proteins by electrophoresis, 5) applying fluorescently-tagged polyclonal antibodies to the gel to bind to a multiplicity of lipoprotein-bound PCSK9 or apolipoprotein moieties on lipid particles in the gel lane, 6) washing the gel to get rid of anything that is not a lipoparticle-antibody complex, 7) detecting all lipoparticles associated with a different fluorescent tag in different bands on the gel, 9) assigning distinct fluorescent signals to PCSK9 and/or different apolipoproteins on lipid particles, 10) obtaining a total concentration number for lipid particles from a different assay such as internal Apo B-related calibration, ultracentrifugation or NMR, 11) calculating the particle numbers based on some combination of optical density/emission coefficients, fluorescent signal wavelength, position in the gel and absolute particle number. Further, in a diagnostic method, one may calculate a ratio of PCSK9 detected in the previous example such as bound PCSK9:total PCSK9, bound PCSK9:free PCSK9, bound PCSK9:LDL, free PCSK9:LDL or bound PCSK9/Apo B, and assess risk for CHD based on the calculated ratio.

In an embodiment, the risk of cardiovascular disease is higher or worsening of cardiovascular disease is assessed when there is a greater level of PCSK9 from baseline.

In an embodiment, the risk of cardiovascular disease is higher or worsening of cardiovascular disease is assessed when the ratio of bound:free PCSK9 is greater than from baseline.

In an embodiment, the risk of cardiovascular disease is higher or worsening of cardiovascular disease is assessed when the ratio of bound PCSK9:Apo B is greater than from baseline.

In an embodiment, the risk of cardiovascular disease is higher or worsening of cardiovascular disease is assessed when the ratio of bound PCSK9:Triglyceride-rich lipoproteins (TRLs) is greater than from baseline.

FIG. 1: A gel comprising two lanes of separated lipoproteins from the same sample generated as described above: it is immunologically labeled with antibody targeted to either Apo B (left) or PCSK9 (right). PCSK9 is observable inside the circles inset into the figure. PCSK9 is found at the same position on the gel as apolipoprotein B, indicating its presence on the same lipoparticles as Apo B. The lack of intensity is a combination of lower concentration analyte PCSK9 concentration is lower than Apo B, and not optimized reagents and methods.

FIGS. 2A-I: In FIGS. 2A-I, multiple zonal gel separations of lipoproteins with distinct detection and visualization protocols are superimposed, resulting in a densitometry scan of PCSK9, Apo B, triglyceride (TG), and cholesterol components on a graph standardized to positions for lipoproteins HDL, VLDL, Lp(a), and LDL. PCSK9 separation and detection is carried out as in the non-specific Lipo-IFE method described above. After separation and immunofixation, non-specific protein stain is used to identify anti-PCSK9 antibodies bound to PCSK9-bound lipoproteins in the gel matrix. Apo B separation and detection is carried out in the same manner, replacing anti-PCSK9 antibody with anti-Apo B antibody. TG and cholesterol portions are separated by the same zonal gel electrophoresis protocol. Detection and visualization are done by incubating the separated lanes with triglyceride visualization reagents. The triglycerides in the sample are hydrolyzed by a combination of microbial lipases to give glycerol and fatty acids. The glycerol is phosphorylated by adenosine triphosphate (ATP) in the presence of glycerol kinase (GK) to produce glycerol-3-phosphate. The glycerol-3-phosphate is oxidized by molecular oxygen in the presence of GPO (glycerol phosphate oxidase) to produce hydrogen peroxide (H202) and dihydroxyacetone phosphate. The formed H202 reacts with 4-aminophenazone and N,N-bis(4-sulfobutyl)-3,5-dimethylaniline, disodium salt (MADB) in the presence of peroxidase (POD) to produce a chromophore, which is read at 660/800 nm. The increase in absorbance at 660/800 nm is proportional to the triglyceride content of the sample. Cholesterol is processed by method known in the art, in general, cholesterol is consumed by cholesterol esterase, cholesterol oxidase, peroxidase and 4-aminoantipyrine to generate a colorless end product, then cholesterol reacts with cholesterol esterase, cholesterol oxidase and a chromogen system to yield a blue color complex which can be measured bichronmatically at 540/660 nm. An example reagent system is available from Beckman Coulter. Concentration of reagents is increased as necessary to achieve visualization.

In FIGS. 2A-I, PCSK9 concentration is represented with the heavy line and calculated in the bottom box. Apo B concentration is presented in the line with no filling and calculated in the second from the bottom box. Cholesterol is visualized with a solid line and the area under its curve is filled in. Triglyceride is illustrated with a solid line and is always lower than Cholesterol concentration.

In the figures, PCSK9 is evident in various particles and in sometimes unexpected positions. The distributions of PCSK9 among lipoproteins is an important measure to compare to cardiovascular health and outcomes.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

I/We claim:

1. A gel electrophoresis system for detecting a level of PCSK9 bound and/or unbound to lipoprotein particles present in a biological sample comprising:
a gel substrate to receive a biological sample; and
an anti-PCSK9 antibody, at least one anti-lipoprotein antibody, or both.

2. An immunoassay system for detecting a level of PCSK9 bound and/or unbound to lipoprotein particles present in a biological sample comprising:
an anti-lipoprotein antibody immobilized on a solid substrate; and
an anti-PCSK9 antibody, wherein the anti-PCSK9 antibody is bound to a distinct signal producing molecule capable of producing or causing production of a distinguishable detectable signal.

3. The system of claim 1 or 2, further comprising a non-specific protein dye.

4. The system of claim 1 or 2, wherein the anti-PCSK9 antibody, at least one anti-lipoprotein antibody, or both are bound to distinct signal producing molecules capable of producing or causing production of a detectable signal and wherein each detectable signal is distinguishable from the other detectable signal(s).

5. The system of claim 1 or 2, further comprising a device for detecting the anti-PCSK9 antibody, the at least one anti-lipoprotein antibody, a detectable signal attached thereto, a non-specific protein dye, or any combination thereof.

6. The system of claim 1 or 2, wherein the anti-lipoprotein antibody is selected from the group consisting of anti-lipoprotein (a), anti-apolipoprotein B (Apo B), and anti-apolipoprotein-a (apo-a).

7. The system of claim 5, wherein the device quantitates the level of specific lipoprotein-bound PCSK9, free PCSK9, PCSK9-free lipoprotein, PCSK9-bound lipoprotein, or any combination thereof based on said detecting.

8. The system of claim 1 or 2, further comprising a reagent capable of interacting with a signal producing molecule, wherein the signal producing molecule produces the detectable signal upon contact with the reagent.

9. The system of claim 4, wherein the detectable signal is detectable by fluorometric means.

10. The system of claim 1 or 2, wherein systems are non-denaturing.

11. A method for measuring improvement or worsening of a patient undergoing treatment for hypercholesterolemia, said method comprising:
obtaining a biological sample from the patient,
measuring the amount of lipoprotein-bound PCSK9, free PCSK9, or both in the biological sample;
measuring the amount of lipoprotein-bound Apo B, free Apo B, or both in the biological sample;
optionally measuring an amount of triglyceride-rich lipoprotein (TRL) in the biological sample;
generating a ratio of bound:free PCSK9, of bound PCSK9:Apo B, and/or of bound PCSK9:TRL; and
comparing the ratio(s) generated to a reference level(s).

12. The method of claim 11, further comprising evaluating the level of improvement or worsening in hypercholesterolemia of the patient by a decreasing or increasing ratio.

13. The method of claim 11, wherein worsening of the patient undergoing treatment for hypercholesterolemia is determined when there is a greater level of PCSK9 than the reference level, the ratio of bound:free PCSK9 is greater than the reference level, the ratio of bound PCSK9:Apo B is greater than the reference level, or the ratio of bound PCSK9:TRLs is greater than the reference level.

14. A method for assessing risk of a patient developing cardiovascular disease or progression of cardiovascular disease, said method comprising:
obtaining a biological sample from the patient,
measuring an amount of lipoprotein-bound PCSK9, free PCSK9, or both in the biological sample;
measuring an amount of lipoprotein-bound Apo B, free Apo B, or both in the biological sample;
optionally measuring an amount of triglyceride-rich lipoprotein (TRL) in the biological sample;
generating a ratio of bound:free PCSK9, of bound PCSK9:Apo B, and/or of bound PCSK9:TRL; and
comparing the ratio(s) to a reference level(s).

15. The method of claim 14, further comprising evaluating the level of risk of developing or progression of cardiovascular disease by a decreasing or increasing ratio.

16. The method of claim 14, wherein the risk of developing cardiovascular disease or progression of cardiovascular disease is determined to be higher or worsening when there is a greater level of PCSK9 than the reference level, the ratio of bound:free PCSK9 is greater than the reference level, the ratio of bound PCSK9:Apo B is greater than the reference level, or the ratio of bound PCSK9:TRLs is greater than the reference level.

17. The method of claim 11 or 14, wherein the lipoprotein-bound PCSK9, free PCSK9, or both is measured via an immunoassay comprising at least an anti-PCSK9 detection antibody and a signal-producing molecule physically bound to the anti-PCSK9 detection antibody and quantifying PCSK9 via signal detected from the signal-producing molecule.

18. The method of claim 11 or 14 wherein the lipoprotein-bound Apo B, free Apo B, or both is measured via an immunoassay comprising at least an anti-Apo B detection antibody and a signal-producing molecule physically bound to the anti-Apo B detection antibody and quantifying Apo B via signal detected from the signal-producing molecule.

19. The method of claim 11 or 14, wherein the anti-PCSK9 antibody binds to an epitope of PCSK9 that comprises at least one residue or a plurality of residues consisting of SEQ ID NO 1, SEQ ID NO 2, SEQ ID NO 3.


SEQUENCE LISTING (3 sequences)

SEQ ID NO: 1 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVTHGLLPGFLVKMSGDLLELALKLPHVDYIEEDSSVFAQSIPWNLERITPPRYRADEYQPPDGGSLVEVYLLDTSIQSDHREIEGRYMVTDFENVPEEDGTRFHRQASKCDSHGTHLAGVVSGRDAGVAKGASMRSLRVINCQGKGTVSGTLIGLEFIRKSQLVQPVGPLVVLLPLAGGYSRVINAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTYWSAHSGPTRMATAVARCAPDEELLSCSSFSRSGKRRGERMEAQGGKLVCRAHNAFGGEGVYAIARCCLLPQANCSVHTAPPAEASMGTRYHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 2 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 3 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ